(19) United States
(12) Patent Application Publication
(10) Pub. No.: US 2011/0152194 A1
(43) Pub. Date: Jun. 23, 2011

(54) CHIMERIC NATRIURETIC POLYPEPTIDES AND METHODS FOR INHIBITING CARDIAC REMODELING

(75) Inventors: John C. Burnett, JR., Rochester, MN (US); Horng H. Chen, Rochester, MN (US); Ondrej Lisy, Rochester, MN (US)

(73) Assignee: MAYO FOUNDATION FOR MEDICAL EDUCATION AND RESEARCH

(21) Appl. No.: 12/996,500

(22) PCT Filed: Jun. 3, 2009
(86) PCT No.: PCT/US2009/046095

§ 371 Date: Mar. 11, 2011

Related U.S. Application Data
(60) Provisional application No. 61/059,576, filed on Jun. 6, 2008.

(51) Int. Cl. A61K 38/16; A61P 9/10

(52) U.S. Cl. 514/16.4

(57) ABSTRACT

Materials and methods related to chimeric polypeptides containing the amino acid sequence of CNP and the C-terminal sequence of DNP. The polypeptides are natriuretic and diuretic, GFR enhancing, cardiac unloading, renin inhibiting and less hypotensive when compared to BNP. The polypeptides also inhibit cardiac fibroblast proliferation.

### CROSS-REFERENCE TO RELATED APPLICATIONS

This application claims benefit of priority from U.S. Provisional Application Ser. No. 61/059,576, filed on Jun. 6, 2008.

### STATEMENT AS TO FEDERALLY SPONSORED RESEARCH

This invention was made with government support under grant nos. HL76611-03 and HL36634-20, awarded by the National Institutes of Health. The government has certain rights in the invention.

### TECHNICAL FIELD

This document relates to compositions comprising natriuretic polypeptides, as well as methods for using natriuretic peptides to prevent, reduce, and/or inhibit cardiac remodeling and prevent, reduce, and/or inhibit ischemic injury after myocardial infarction (MI).

### BACKGROUND

The natriuretic peptide family includes the cardiac hormones atrial natriuretic peptide (ANP), B-type natriuretic peptide (BNP), C-type natriuretic peptide (CNP), and Dendroaspis natriuretic peptide (DNP), all of which function via well characterized particulate guanylyl cyclase receptors (i.e., NPR-A for ANP and BNP; NPR-B for CNP) and the second messenger cyclic 3′5′ guanosine monophosphate (cGMP) (Kuhn (2003) Circ Res 93:700-709; Tawaragi et al. (1991) Biochem Biophys Res Commun 175:645-651; and Komatsu et al. (1991) Endocrinology 129:1104-1106). CNP has beneficial vascular and anti-proliferative properties. While lacking renal actions, CNP is less hypotensive than ANP and BNP, but unloads the heart due to venodilation. DNP is a potent natriuretic and diuretic peptide that is markedly hypotensive. CNP and DNP function via separate guanylyl cyclase receptors.

### SUMMARY

This document is based in part on the identification of chimeric peptide compositions and methods that combine the beneficial properties of two distinct natriuretic peptides. As described herein, fusion of CNP (GLSKGCFGLKLDRIGSMSGLGC; SEQ ID NO:1) with the 15-AA linear C-terminus of DNP (PSLRDPRPNAPSTSA; SEQ ID NO:2) results in a synthetic chimeric peptide (CD-NP) that in vivo is natriuretic and diuretic, GFR enhancing, cardiac unloading, and renin inhibiting, with minimal hypotensive properties. In addition, as demonstrated by in vitro studies in cardiac fibroblasts (CFs), CD-NP has cGMP activating and anti-proliferative properties. Further, other natriuretic polypeptides and chimeric polypeptides can be used in accordance with the present disclosure. The findings disclosed herein advance an innovative design strategy in natriuretic peptide drug discovery and development to create therapeutic peptides with properties that may be preferable to those associated with native natriuretic peptides, and that have potentially beneficial efficacy and safety for treatment of cardiorenal disease states such as acute heart failure (AHF), acute myocardial infarction (AMI), reperfusion injury, ischemic injury, and cardiac remodeling.

In one aspect, this document features a method for reducing cardiac remodeling in a subject identified as being in need thereof, the method comprising administering to the subject a composition comprising a pharmaceutically acceptable carrier and a polypeptide capable of increasing urinary and plasma cGMP levels in the subject, wherein the composition is administered in an amount effective to alter the level of one or more parameters of cardiac remodeling by at least ten percent as compared to the levels of the one or more parameters prior to administering the composition, and wherein the one or more parameters are selected from the group consisting of cardiac unloading, increased glomerular filtration rate, decreased levels of aldosterone, decreased plasma renin activity, decreased levels of angiotensin II, decreased proliferation of cardiac fibroblasts, decreased left ventricular mass, decreased left ventricular hypertrophy, decreased ventricular fibrosis, increased ejection fraction, decreased left ventricular end systolic diameter, decreased pulmonary wedge capillary pressure, decreased right atrial pressure, and decreased mean arterial pressure.

The polypeptide can be a natriuretic polypeptide. The natriuretic polypeptide can be a chimeric natriuretic polypeptide comprising (a) the ring structure of a first natriuretic polypeptide or a variant of the ring structure of the first natriuretic polypeptide, and (b) an amino acid sequence from a second natriuretic polypeptide or a variant of the amino acid sequence from the second natriuretic polypeptide. The natriuretic polypeptide can comprise the amino acid sequence set forth in SEQ ID NO:3, but with one, two, three, four, or five amino acid substitutions relative to the sequence set forth in SEQ ID NO:3. The polypeptide can be capable of binding to the NPR-B receptor and the NRP-A receptor. The polypeptide can have an elimination half-life of at least 15 minutes after administration to the subject.

The method can comprise administering the composition as a continuous intravenous infusion (e.g., for one to seven days). The method can comprise administering the composition as a continuous intravenous infusion for one to seven days, and subsequently administering the composition subcutaneously for five to 30 days. The method can comprise administering the composition as a continuous intravenous infusion at a dose of about 0.1 ng polypeptide/kg body mass/minute to about 30 ng polypeptide/kg body mass/minute, and subsequently administering the composition subcutaneously at a dose of about 10 ng polypeptide/kg body mass/day to about 30 ng polypeptide/kg body mass/day. The method can comprise administering the composition as a continuous intravenous infusion at a dose of about 0.1 ng polypeptide/kg body mass/minute to about 30 ng polypeptide/kg body mass/minute for about three hours to about seven days, and subsequently administering the composition subcutaneously at a dose of about 10 ng polypeptide/kg body mass/day to about 30 ng polypeptide/kg body mass/day for about five to about 30 days.

The subject can be identified as having acute heart failure or acute myocardial infarction. The method can comprise administering the continuous intravenous infusion beginning at or about the time of reperfusion, or beginning about three hours after the onset of reperfusion. The composition can be administered from about three hours to about 12 hours after reperfusion. The method can comprise administering the composition at a dose of about 1 ng polypeptide/kg body mass/minute to about 30 ng polypeptide/kg body mass/minute (e.g., about 10 ng polypeptide/kg body mass/minute, about 12.5 ng polypeptide/kg body mass/minute, about 15 ng polypeptide/kg body mass/minute, about 17.5 ng polypeptide/kg body mass/minute, or about 20 ng polypeptide/kg body mass/minute). The method can further comprise monitoring the subject for the level of one or more parameters of cardiac remodeling.

In another aspect, this document features a composition comprising a pharmaceutically acceptable carrier and a polypeptide, wherein the polypeptide is capable of increasing urinary and plasma cGMP levels in a subject, wherein the composition, when administered to a subject identified as being in need thereof, results in reduced cardiac remodeling, wherein the reduced or inhibited cardiac remodeling is indicated by an alteration in the levels of one or more parameters selected from the group consisting of cardiac unloading, increased glomerular filtration rate, decreased levels of aldosterone, decreased plasma renin activity, decreased levels of angiotensin II, decreased proliferation of cardiac fibroblasts, decreased left ventricular mass, decreased left ventricular hypertrophy, decreased ventricular fibrosis, increased ejection fraction, decreased left ventricular end systolic diameter, decreased pulmonary wedge capillary pressure, decreased right atrial pressure, and decreased mean arterial pressure, and wherein the levels of the one or more parameters are altered by at least ten percent as compared to the levels of the one or more parameters prior to the administration.

The polypeptide can be a natriuretic polypeptide. The natriuretic polypeptide can be a chimeric natriuretic polypeptide comprising (a) the ring structure of first natriuretic polypeptide or a variant of the ring structure of the first natriuretic polypeptide, and (b) an amino acid sequence from a second natriuretic polypeptide or a variant of the amino acid sequence from the second natriuretic polypeptide. The natriuretic polypeptide can comprise the amino acid sequence set forth in SEQ ID NO:3, but with one, two, three, four, or five amino acid substitutions relative to the sequence set forth in SEQ ID NO:3. The polypeptide can be capable of binding to the NPR-B receptor and the NRP-A receptor. The polypeptide can have an elimination half-life of at least 15 minutes after administration to a subject. The natriuretic polypeptide can comprise an amino acid sequence that is between 91 and 98 percent identical to the amino acid sequence set forth in SEQ ID NO:3. The natriuretic polypeptide can comprise the amino acid sequence of SEQ ID NO:3, but with one, two, three, four, or five amino acid substitutions relative to the sequence set forth in SEQ ID NO:3. The subject can be identified as having acute heart failure or acute myocardial infarction. The pharmaceutical carrier can be normal saline or dextrose and water.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a diagram showing the amino acid sequences and structures of CNP (SEQ ID NO:1), DNP (SEQ ID NO:17), the 37-AA chimeric natriuretic peptide, CD-NP (SEQ ID NO:3), and the 27-AA “transformed” CNP referred to as CNP-C (SEQ ID NO:4).

FIG. 2 is a pair of graphs showing an example of the effect of C-terminus of DNP on urinary excretion of sodium (UNaV; FIG. 2A) and urine flow (UV; FIG. 2B) in normal dogs (n=6). Data are expressed as means±SE. C-terminus, intravenous infusion of 42 ng/kg/min of C-terminus. *P<0.05 vs. baseline.

FIG. 3 is a series of graphs showing an example of the effect of CD-NP on mean arterial pressure (MAP; FIG. 3A), right atrial pressure (RAP; FIG. 3B) and pulmonary capillary pressure (PCWP; FIG. 3C) in normal dogs (n=6). Data are expressed as means±SE. CD-NP 10, dose of CD-NP 10 ng/kg/min; CD-NP 50, dose of CD-NP 50 ng/kg/min; CD-NP 100, dose of CD-NP 100 ng/kg/min. *P<0.05 vs. baseline.

FIG. 4 is a series of graphs showing an example of the effect of CD-NP on urinary excretion of sodium (UNaV; FIG. 4A), urine flow (UV; FIG. 4B) and glomerular filtration rate (GFR; FIG. 4C) in normal dogs (n=6). Data are expressed as means±SE. CD-NP 10, dose of CD-NP 10 ng/kg/min; CD-NP 50, dose of CD-NP 50 ng/kg/min; CD-NP 100, dose of CD-NP 100 ng/kg/min. *P<0.05 vs. baseline.

FIG. 5 is a pair of graphs showing an example of the effect of CD-NP on proximal fractional sodium reabsorption (PFRNa; FIG. 5A) and distal fractional sodium reabsorption (DFRNa; FIG. 5B) in normal dogs (n=6). Data are expressed as means±SE. CD-NP 10, dose of CD-NP 10 ng/kg/min; CD-NP 50, dose of CD-NP 50 ng/kg/min; CD-NP 100, dose of CD-NP 100 ng/kg/min. *P<0.05 vs. baseline.

FIG. 6 is a pair of graphs showing an example of the effect of two doses of CD-NP and equimolar doses of human BNP in two groups of normal dogs (n=6 in each group). FIG. 6A illustrates the effect of CD-NP on mean arterial pressure (MAP), and FIG. 6B illustrates the effect of CD-NP on glomerular filtration rate (GFR). Filled bars, CD-NP; empty bars, BNP. Data are expressed as means±SE. CD-NP 10, dose of CD-NP 10 ng/kg/min or an equimolar dose of BNP in other group; CD-NP 50, dose of CD-NP 50 ng/kg/min or an equimolar dose of BNP in other group.*P<0.05 vs. baseline. †P<0.05 between groups.

FIG. 7 is a pair of graphs showing an example of the effects of CD-NP in human CFs. FIG. 7A illustrates the effect of CD-NP on cGMP generation in human CFs. Data are expressed as means±SE. *P<0.05 vs. no treatment; **P<0.05 vs. CP-NP 10⁻¹¹ M; +P<0.05 vs. CD-NP 10⁻⁸ M. FIG. 7B shows an example of the antiproliferative actions of CD-NP in human CFs, as plotted by BrdU uptake in optical density units as measured by colorimetry. Control, untreated human cardiac fibroblasts; Cardiotrophin-1, proliferation of fibroblasts by Cardiotrophin-1; Cardiotrophin-1+CD-NP, CD-NP added to Cardiotrophin-1 stimulated fibroblasts. Data are expressed as means±SE. *P<0.05 vs. control.

FIG. 8 is a graph showing left ventricular (LV) mass in rats three weeks after myocardial infarction (MI) in one embodiment. MI, no treatment; MI+CDNP, treatment with 1.7×10⁻⁷ g/kg/minute CD-NP for two weeks after MI.

FIG. 9A is a series of graphs showing plasma cGMP levels (left panel), urinary cGMP excretion (middle panel), and net renal generation of cGMP (right panel) in dogs treated with CD-NP or CNP, as indicated. Values are means±SEM (for CD-NP, n=9-10; for CNP, n=7-9). Comparisons within group vs. pre-infusion (mean±SEM, P<0.05*, P<0.01†) and between groups (P<0.001 [symbol] ) were made. Time denotes mid-clearance. FIG. 9B is a pair of graphs showing urinary flow (left panel) and urinary sodium excretion (right panel) in dogs treated with CD-NP or CNP, as indicated. Values are means±SEM. Comparisons within group vs. pre-infusion (mean±SEM, P<0.05*, P<0.01†) and between groups (P<0.05‡, P<0.001 [symbol] ) were made (for CD-NP, n=10; for CNP, n=7). Time denotes mid-clearance.

FIGS. 10A-10D are graphs showing plasma cGMP (FIG. 10A), urinary cGMP excretion (FIG. 10B), natriuretic response (FIG. 10C), and blood pressure response (FIG. 10D) in humans treated with CD-NP or placebo, as indicated. Comparisons within group vs. pre-infusion (mean±SEM, P<0.05*, P<0.01†) and between groups (P=0.01 [symbol] ) were made. FIG. 10E is a graph showing the cGMP response to CD-NP in the absence or presence of an NPR-A antagonist (1 μM) in isolated canine glomeruli. *P<0.05 vs. blank, †P<0.0001 vs. blank.

### DETAILED DESCRIPTION

### Compounds

This document provides natriuretic compounds and compositions that can be used to increase cGMP levels and reduce cardiac remodeling in a subject in need thereof. As described herein, the compounds can bind to the NPR-A receptor and/or the NPR-B receptor and, in some cases, the NPR-C receptor. In addition, the compounds can have longer elimination half-lives than native NPs after administration to a subject. In some embodiments, the compounds provided herein can be polypeptides.

For example, this document describes exemplary isolated natriuretic polypeptides that are capable of inhibiting or reducing AHF, AMI, reperfusion injury, ischemic injury, and cardiac remodeling. In some embodiments, natriuretic peptides can be used to treat, inhibit, and/or prevent cardiac remodeling and ischemia injury, particularly after AMI and/or AHF. As used herein, the term “natriuretic polypeptide” or “NP” includes native (naturally occurring, wild type) NPs (e.g., ANP, BNP, CNP, DNP, and urodilatin), one or more portions of a native NP, variants of a native NP, or chimeras of native NPs, portions of native NPs, or variants of native NPs or portions of native NPs. In some embodiments, a NP includes only portions of the mature form of a native NP. NPs containing amino acid sequences from CNP and DNP can be particularly useful, although other native and chimeric NPs are contemplated herein.

CNP is a 22-amino acid peptide that shares structural homology with but is genetically distinct from ANP and BNP. Also unlike ANP or BNP, CNP lacks a C-terminal amino acid extension, which may explain in part its lack of natriuretic properties (Clavell et al. (1993) Am Heart J 1104-1106; and Hunt et al. (1994) J Clin Endocrinol Metab 78:1428-1435). CNP is principally an endothelial cell-derived peptide (Stingo et al. (1992) Am Heart J H1318-1321; Ogawa et al. (1992) Hypertension 19:809-813; Doi et al. (2001) Arterioscler Thromb Vasc Biol 21:930-936; Naruko et al. (2005) Atherosclerosis 181:241-250; Horio et al. (2003) Endocrinology 144:2279-2284; Langenickel et al. (2006) Proc Natl Acad Sci USA 103:4735-4740; and Scotland et al. (2005) Proc Natl Acad Sci USA 102:14452-14457). In isolated venous and arterial rings, CNP activates NPR-B receptors in veins, while ANP and BNP bind to NPR-A receptors in both arteries and veins. This is consistent with the less hypotensive actions of CNP as compared to ANP and BNP (Wei et al. (1993) Am J Physiol 264:H71-73; Igaki et al. (1998) Hypertens Res 21:7-13; and La Villa et al. (1998) Clin Sci (Lond) 95:595-602).

In addition to venodilating properties, CNP has more potent anti-proliferative and collagen suppressing properties in CFs as compared to ANP and BNP (Horio et al., supra). For example, studies have shown that 14 days of continuous infusion of CNP in rodents with AMI markedly attenuates ventricular dilatation, cardiac fibrosis, and cardiomyocyte hypertrophy (Soeki et al. (2005) J Am Coll Cardiol 45:608-616). Chronic infusion of CNP did not result in hypotensive actions.

In contrast to ANP and BNP, CNP lacks significant natriuretic and diuretic actions when infused into humans. This may explain its lack of utility in sodium and water retaining syndromes such as AHF, despite its attractive venodilating and anti-fibrotic properties (Igaki et al., supra; and La Villa et al., supra).

DNP was originally isolated from the green mamba. DNP is potently natriuretic and diuretic in vivo, and possesses cardiac unloading actions but with significant hypotensive properties (Schweitz et al. (1992) J Biol Chem 267:13928-13932; Lisy et al. (1999) Kidney Int 56:502-508; and Lisy et al. (2001) Hypertension 37:1089-1094). DNP, like ANP and BNP, functions via the NPR-A receptor, as saturation with ANP markedly attenuates the cGMP activating actions of DNP in cultured human endothelial cells. Indeed, the in vivo actions of DNP that include natriuresis and hypotension are consistent with NPR-A activation, as such effects closely mimic the properties of ANP and BNP but not CNP. In fact, DNP has been shown to have a higher affinity for the NPR-A receptor in human myocardium as compared to ANP and BNP (Singh et al. (2006) Circ Res 99:183-190).

DNP has the longest C-terminus of the known natriuretic peptides, consisting of 15-AA as compared to 5-AA for ANP, 6-AA for BNP, and none for CNP. The long C-terminus of DNP may render DNP highly resistant to degradation by neutral endopeptidase (NEP), thus contributing to its potent natriuretic and diuretic actions (Chen et al. (2002) J Am Coll Cardiol 40:1186-1191). Further, the lack of a C-terminus for CNP may explain the observation that of the three known endogenous natriuretic peptides, CNP is the most susceptible to NEP degradation. The lack of a C-terminus also could explain CNP's renal actions, as NEP is most highly expressed in the kidney (Kenny and Stephenson (1988) FEBS Lett 232:1-8).

Urodilatin is an ANP-like agonist of the NPR-A receptor having potent natriuretic and diuretic activity. Urodilatin is localized in the kidney, is differentially processed from the same precursor as ANP, and is secreted into the urine. The 32 amino acid sequence of urodilatin includes the entire 28 amino acid sequence of ANP, with a four amino acid extension at its N-terminus.

The term “cardiac remodeling” refers to effects on the heart that can occur with MI, AHF, or other conditions. These include, for example, heart dilation, myocyte hypertrophy, and cardiofibrosis (i.e., proliferation of interstitial fibroblasts). The NPs provided herein can inhibit or prevent cardiac remodeling that occurs with AMI or AHF. In some embodiments, parameters indicative of reduced cardiac remodeling can include one or more of the following: cardiac unloading (i.e., reduced pressure in the heart), increased glomerular filtration rate (GFR), decreased plasma renin activity (PRA), decreased levels of angiotensin II, decreased proliferation of cardiac fibroblasts, decreased left ventricular (LV) hypertrophy, decreased LV mass (indicative of reduced fibrosis and hypertrophy), decreased pulmonary wedge capillary pressure (PWCP; an indirect measure of left atrial pressure), decreased right atrial pressure, decreased mean arterial pressure, decreased levels of aldosterone (indicative of an anti-fibrotic effect), decreased ventricular fibrosis, increased ejection fraction, and decreased LV end systolic diameter. To determine whether a NP is capable of inhibiting or reducing cardiac remodeling, one or more of these parameters can be evaluated (e.g., before and after treatment with the NP), using methods known in the art and/or described herein, for example.

Conditions such as AMI and AHF can lead to kidney damage as well as heart damage. In some embodiments, the NPs provided herein also can protect the kidneys from damage after AMI and AHF. Parameters that are indicative of kidney protection include, for example, decreased proximal fractional reabsorption of sodium (PFRNa), decreased distal fractional reabsorption of sodium (DFRNa), increased urinary sodium excretion (UNaV), and increased urine flow (UV). Any one or more of these parameters can be assessed (e.g., before and after administration of a NP) to determine whether the NP has kidney protecting effects. Methods for assessing these parameters are known in the art, and also are described herein.

The term “isolated polypeptide” refers to a polypeptide that (1) is not associated with proteins found in nature, (2) is free of other proteins from the same source (e.g., free of human proteins), (3) is expressed by a cell from a different species, or (4) does not occur in nature. The isolated polypeptides provided herein typically contain 10 or more (e.g., 12 or more, 15 or more, or 20 or more) amino acid residues. An isolated polypeptide can be, for example, encoded by DNA or RNA, including synthetic DNA or RNA, or some combination thereof.

Chimeric NPs can include amino acid sequences from two or more individual NPs. In some embodiments, a chimeric polypeptide can include amino acid sequences from CNP and DNP. In addition, in some cases a chimeric NP can include a ring structure and cysteine bond (e.g., the ring structure and cysteine bond of ANP, BNP, CNP, or DNP) in combination with one or more amino acid segments from another NP. For example, a chimeric CD-NP can include the entire 22-AA sequence of CNP (GLSKGCFGLKLDRIGSMSGLGC; SEQ ID NO:1) and the 15-AA C-terminus of DNP (PSLRDPRPNAPSTSA; SEQ ID NO:2), and thus can have the amino acid sequence set forth in SEQ ID NO:3 (GLSKGCFGLKLDRIGSMSGLGCPSLRDP RPNAPSTSA). In some embodiments, a chimeric NP can include amino acid sequences from CNP and urodilatin. For example, a chimeric CU-NP can include the ring structure and disulfide bond of CNP (CFGLKLDRIGSMSGLGC; SEQ ID NO:5) in combination with the ten amino acid N-terminus (TAPRSLRRSS; SEQ ID NO:6) and the five amino acid C-terminus (NSFRY; SEQ ID NO:7) of urodilatin, and thus can have the sequence TAPRSLRRSSCFGLKLDRIGSMSGLGCNSFRY (SEQ ID NO:8).

In some cases, a chimeric NP can include a variant (e.g., a substitution, addition, or deletion) at one or more positions (e.g., one, two, three, four, five, six, seven, eight, nine, or ten positions) with respect to SEQ ID NO:3 or SEQ ID NO:8. Variant NPs, e.g., those having one or more amino acid substitutions relative to a native NP amino acid sequence, can be prepared and modified as described herein. Amino acid substitutions can be made, in some cases, by selecting substitutions that do not differ significantly in their effect on maintaining (a) the structure of the peptide backbone in the area of the substitution, (b) the charge or hydrophobicity of the molecule at the target site, or (c) the bulk of the side chain. For example, naturally occurring residues can be divided into groups based on side-chain properties: (1) hydrophobic amino acids (norleucine, methionine, alanine, valine, leucine, and isoleucine); (2) neutral hydrophilic amino acids (cysteine, serine, and threonine); (3) acidic amino acids (aspartic acid and glutamic acid); (4) basic amino acids (asparagine, glutamine, histidine, lysine, and arginine); (5) amino acids that influence chain orientation (glycine and proline); and (6) aromatic amino acids (tryptophan, tyrosine, and phenylalanine). Substitutions made within these groups can be considered conservative substitutions. Non-limiting examples of useful substitutions include, without limitation, substitution of valine for alanine, lysine for arginine, glutamine for asparagine, glutamic acid for aspartic acid, serine for cysteine, asparagine for glutamine, aspartic acid for glutamic acid, proline for glycine, arginine for histidine, leucine for isoleucine, isoleucine for leucine, arginine for lysine, leucine for methionine, leucine for phenyalanine, glycine for proline, threonine for serine, serine for threonine, tyrosine for tryptophan, phenylalanine for tyrosine, and/or leucine for valine.

Non-limiting examples of variant CD-NP include the following:

(SEQ ID NO: 9)
PLSKGCFGLKLDRIGSMSGLGCPSLRDPRPNAPSTSA,
(SEQ ID NO: 10)
GISKGCFGLKLDRIGSMSGLGCPSLRDPRPNAPSTSA,
(SEQ ID NO: 11)
GLSKGCFGLKLDRIGSMSGLGCPSLRDPRPNAPSTTA,
(SEQ ID NO: 12)
GLSKGCFGLKLDRIGSMSGLGCPSLRDPRPNAPSTSV,
(SEQ ID NO: 13)
GLTKGCFGLKLDRIGSMSGLGCPSLRDPRPNAPSTSA,
(SEQ ID NO: 14)
GLSRGCFGLKLDRIGSMSGLGCPSLRDPRPNAPSTSA,
(SEQ ID NO: 15)
GLSKGCFGLKLDRIGSMSGLGCPSLRDPRPNAPSSSA,
and
(SEQ ID NO: 16)
GLSKGCFGLKLDRIGSMSGLGCPSLRDPRPNAPTTSA.

Further examples of conservative substitutions that can be made at any position within CD-NP are set forth in Table 1.

TABLE 1
Examples of conservative amino acid substitutions

| Original | Exemplary | Preferred |
|---|---|---|
| Residue | substitutions | substitutions |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn | Asn |
| Glu | Asp | Asp |
| Gly | Pro | Pro |
| His | Asn, Gln, Lys, Arg | Arg |
| Ile | Leu, Val, Met, Ala, Phe, Norleucine | Leu |
| Leu | Norleucine, Ile, Val, Met, Ala, Phe | Ile |
| Lys | Arg, Gln, Asn | Arg |
| Met | Leu, Phe, Ile | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleucine | Leu |

In some embodiments, a NP can include one or more non-conservative substitutions. Non-conservative substitutions typically entail exchanging a member of one of the classes described above for a member of another class. Such production can be desirable to provide large quantities or alternative embodiments of such compounds. Whether an amino acid change results in a functional polypeptide can readily be determined by assaying the specific activity of the peptide variant.

Variant NPs having conservative and/or non-conservative substitutions (e.g., with respect to SEQ ID NO:3), as well as fragments of SEQ ID NO:3, fragments of variants of SEQ ID NO:3, and polypeptides comprising SEQ ID NO:3, variants or fragments of SEQ ID NO:3, or fragments of variants of SEQ ID NO:3, can be screened for biological activity any suitable assays, including those described herein. For example, the activity of a NP as described herein can be evaluated in vitro by measuring its effect on cGMP levels generated by CFs or by testing its ability to suppress proliferation of CFs, as described in Examples 1 and 3 herein. The activity of a NP also can be evaluated in vivo by, for example, testing its effects on factors such as pulmonary capillary wedge pressure, right atrial pressure, mean arterial pressure, urinary sodium excretion, urine flow, proximal and distal fractional sodium reabsorption, plasma renin activity, plasma and urinary cGMP levels, glomerular filtration rate, and left ventricular mass in animals after induced MI. Such assays are described, e.g., in Examples 1, 2, and 4 herein.

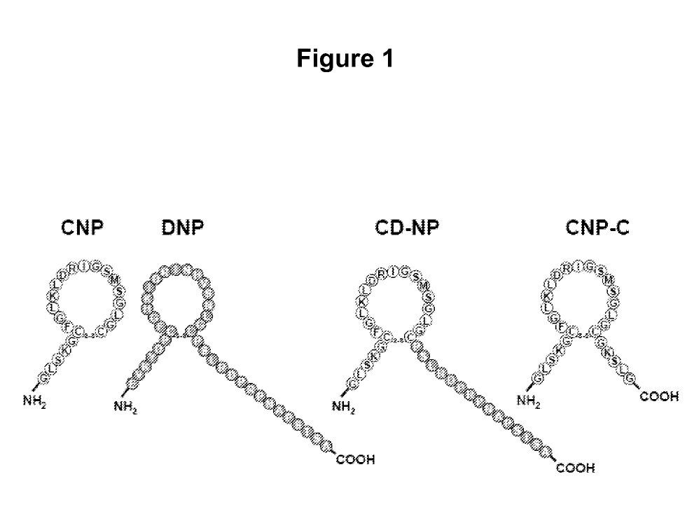

In some embodiments, the NPs provided herein can be cyclic due to disulfide bonds between cysteine residues (see, e.g., the CD-NP structure depicted in FIG. 1). In some embodiments, a sulfhydryl group on a cysteine residue can be replaced with an alternative group (e.g., —CH₂CH₂—). To replace a sulfhydryl group with a —CH₂— group, for example, a cysteine residue can be replaced by alpha-aminobutyric acid. Such cyclic analog polypeptides can be generated, for example, in accordance with the methodology of Lebl and Hruby (Tetrahedron Lett., 1984, 25:2067), or by employing the procedure disclosed in U.S. Pat. No. 4,161,521.

In addition, ester or amide bridges can be formed by reacting the OH of serine or threonine with the carboxyl group of aspartic acid or glutamic acid to yield a bridge having the structure —CH₂CO₂CH₂—. Similarly, an amide can be obtained by reacting the sidechain of lysine with aspartic acid or glutamic acid to yield a bridge having the structure —CH₂C(O)NH(CH)₄—. Methods for synthesis of these bridges are known in the art (see, e.g., Schiller et al. (1985) Biochem. Biophy. Res. Comm. 127:558, and Schiller et al. (1985) Int. J. Peptide Protein Res. 25:171). Other bridge-forming amino acid residues and reactions are provided in, for example, U.S. Pat. No. 4,935,492. Preparation of peptide analogs that include non-peptidyl bonds to link amino acid residues also are known in the art. See, e.g., Spatola et al. (1986) Life Sci. 38:1243; Spatola (1983) Vega Data 1(3); Morley (1980) Trends Pharm. Sci. 463-468; Hudson et al. (1979) Int. J. Pept. Prot. Res. 14:177; Spatola, in Chemistry and Biochemistry of Amino Acid Peptides and Proteins, B. Weinstein, ed., Marcel Dekker, New York, p. 267 (1983); Hann (1982) J. Chem. Soc. Perkin Trans. 1:307; Almquist et al. (1980) J. Med. Chem. 23:1392; Jennings-White et al. (1982) Tetrahedron Lett. 23:2533; European Patent Application EP 45665; Holladay et al. (1983) Tetrahedron Lett. 24:4401; and Hruby (1982) Life Sci. 31:189.

In some embodiments, a NP can comprise an amino acid sequence as set forth in SEQ ID NO:3, but with a particular number of amino acid substitutions. For example, a NP can have the amino acid sequence of SEQ ID NO:3 with one, two, three, four, or five amino acid substitutions. Examples of such amino acid sequences include, without limitation, those set forth in SEQ ID NOS:9-16.

In some embodiments, a NP as provided herein can have an amino acid sequence with at least 85% (e.g., 85%, 86%, 87%, 88%, 89%, 90%, 91%, 92%, 93%, 94%, 95%, 96%, 97%, 97.5%, 98%, 98.5%, 99.0%, 99.5%, 99.6%, 99.7%, 99.8%, 99.9%, or 100%) sequence identity with a region of a reference NP sequence (e.g., SEQ ID NO:1, SEQ ID NO:2, or SEQ ID NO:3). Percent sequence identity is calculated by determining the number of matched positions in aligned amino acid sequences, dividing the number of matched positions by the total number of aligned amino acids, and multiplying by 100. A matched position refers to a position in which identical amino acids occur at the same position in aligned amino acid sequences. Percent sequence identity also can be determined for any nucleic acid sequence.

Percent sequence identity is determined by comparing a target nucleic acid or amino acid sequence to the identified nucleic acid or amino acid sequence using the BLAST 2 Sequences (Bl2seq) program from the stand-alone version of BLASTZ containing BLASTN version 2.0.14 and BLASTP version 2.0.14. This stand-alone version of BLASTZ can be obtained on the World Wide Web from Fish & Richardson's web site (fr.com/blast) or the U.S. government's National Center for Biotechnology Information web site (ncbi.nlm.nih.gov). Instructions explaining how to use the Bl2seq program can be found in the readme file accompanying BLASTZ.

Bl2seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to −1; -r is set to 2; and all other options are left at their default setting. The following command will generate an output file containing a comparison between two sequences: C:\Bl2seq -i c:\seq1.txt -j c:\seq2.txt -p blastn -o c:\output.txt -q −1 -r 2. If the target sequence shares homology with any portion of the identified sequence, then the designated output file will present those regions of homology as aligned sequences. If the target sequence does not share homology with any portion of the identified sequence, then the designated output file will not present aligned sequences.

Once aligned, a length is determined by counting the number of consecutive nucleotides from the target sequence presented in alignment with sequence from the identified sequence starting with any matched position and ending with any other matched position. A matched position is any position where an identical nucleotide is presented in both the target and identified sequence. Gaps presented in the target sequence are not counted since gaps are not nucleotides. Likewise, gaps presented in the identified sequence are not counted since target sequence nucleotides are counted, not nucleotides from the identified sequence.

The percent identity over a particular length is determined by counting the number of matched positions over that length and dividing that number by the length followed by multiplying the resulting value by 100. For example, if (1) a target sequence that is 30 amino acids in length is compared to the sequence set forth in SEQ ID NO:3, (2) the Bl2seq program presents 27 amino acids from the target sequence aligned with a region of the sequence set forth in SEQ ID NO:3 where the first and last amino acids of that 27 amino acid region are matches, and (3) the number of matches over those 27 aligned amino acids is 25, then the 30 amino acid target sequence contains a length of 27 and a percent identity over that length of 92.6 (i.e., 25÷27×100=92.6).

It will be appreciated that different regions within a single amino acid or nucleic acid target sequence that aligns with an identified sequence can each have their own percent identity. It is noted that the percent identity value is rounded to the nearest tenth. For example, 78.11, 78.12, 78.13, and 78.14 are rounded down to 78.1, while 78.15, 78.16, 78.17, 78.18, and 78.19 are rounded up to 78.2. It also is noted that the length value will always be an integer.

Isolated polypeptides can be produced using any suitable methods, including solid phase synthesis, and can be generated using manual techniques or automated techniques (e.g., using an Applied BioSystems (Foster City, Calif.) Peptide Synthesizer or a Biosearch Inc. (San Rafael, Calif.) automatic peptide synthesizer. Disulfide bonds between cysteine residues can be introduced by mild oxidation of the linear polypeptides using KCN as taught, e.g., in U.S. Pat. No. 4,757,048. NPs also can be produced recombinantly, as described below.

Salts of carboxyl groups of polypeptides can be prepared by contacting the peptide with one or more equivalents of a desired base such as, for example, a metallic hydroxide base (e.g., sodium hydroxide), a metal carbonate or bicarbonate base (e.g., sodium carbonate or sodium bicarbonate), or an amine base (e.g., triethylamine, triethanolamine, and the like). Acid addition salts of polypeptides can be prepared by contacting the polypeptide with one or more equivalents of an inorganic or organic acid (e.g., hydrochloric acid).

Esters of carboxyl groups of polypeptides can be prepared using any suitable means (e.g., those known in the art) for converting a carboxylic acid or precursor to an ester. For example, one method for preparing esters of the present polypeptides, when using the Merrifield synthesis technique, is to cleave the completed polypeptide from the resin in the presence of the desired alcohol under either basic or acidic conditions, depending upon the resin. The C-terminal end of the polypeptide then can be directly esterified when freed from the resin, without isolation of the free acid.

Amides of polypeptides can be prepared using techniques (e.g., those known in the art) for converting a carboxylic acid group or precursor to an amide. One method for amide formation at the C-terminal carboxyl group includes cleaving the polypeptide from a solid support with an appropriate amine, or cleaving in the presence of an alcohol, yielding an ester, followed by aminolysis with the desired amine.

N-acyl derivatives of an amino group of a polypeptide can be prepared by utilizing an N-acyl protected amino acid for the final condensation, or by acylating a protected or unprotected peptide. O-acyl derivatives can be prepared for example, by acylation of a free hydroxy peptide or peptide resin. Either acylation may be carried out using standard acylating reagent such as acyl halides, anhydrides, acyl imidazoles, and the like. Both N- and O-acylation may be carried out together, if desired.

In some embodiments, the NPs provided herein can have half-lives that are increased relative to the half-life of native NPs. For example, while the half-life of CNP is short (about a minute and a half), the elimination half-life of CD-NP after administration to a mammal is about 18.5 minutes (see, e.g., Lee et al. BMC Pharmacol. (2007) 7(Suppl. 1):P38; and Lee et al. J. Cardiac Failure (2007) 13(6 Suppl.):S144). Thus, a NP provided herein can have a half life that is increased by at least 2-fold (e.g., at least 2-fold, at least 3-fold, at least 4-fold, at least 5-fold, at least 6-fold, at least 7-fold, at least 8-fold, at least 9-fold, or at least 10-fold) as compared to a native NP such as CNP, for example. In some embodiments, a NP can have an elimination half-life of at least about 10 minutes (e.g., at least about 10 minutes, at least about 12 minutes, at least about 15 minutes, at least about 17 minutes, at least about 18 minutes, or at least about 20 minutes).

A NP as provided herein can function through the one or more of the guanylyl cyclase receptors through which the native NPs function. For example, in some embodiments, a NP as provided herein can bind to and function through the NPR-A receptor through which ANP and BNP function. In some cases, a NP can bind to and function through the NPR-A receptor, as do ANP and BNP. In some cases, a NP as provided herein can function through the NPR-B receptor through which CNP functions. In some cases, a NP as provided herein can bind to and function through the NPR-C receptor. Further, in some cases, a NP as provided herein (e.g., a chimeric NP such as CD-NP) can bind to and function through more than one guanylyl cyclase receptor, including NPR-A and NPR-B, for example. Methods for evaluating which receptor is involved in function of a particular NP are known in the art, and include those set forth in Example 5 herein.

The compounds (e.g., isolated NPs) provided herein can inhibit or reduce cardiac remodeling such as occurs after AMI or AHF, for example. A compound that can inhibit cardiac remodeling is one that can alter one or more parameters indicative of inhibited or reduced cardiac remodeling by at least 10%, as described below. To determine whether a particular compound has such properties, one can carry out assays that are well known to the art, including those described herein. Compounds that are variant NPs typically have at least about 10% (e.g., at least about 10%, 15%, 20%, 25%, 33%, 40%, 50%, 60%, 67%, 75%, 80%, 85%, 90%, 95%, 100%, or more than 100%) of the biological activity of the corresponding wild type NP or, if the NP in question is a chimeric NP (e.g., CD-NP), of the corresponding chimeric NP containing the wild type sequence of the NP components included therein.

### Nucleic Acids, Vectors, and Host Cells

This document also describes exemplary nucleic acids encoding polypeptides (e.g., NPs), as well as expression vectors containing the nucleic acids, and host cells containing the nucleic acids and/or expression vectors. As used herein, the term “nucleic acid” refers to both RNA and DNA, including cDNA, genomic DNA, and synthetic (e.g., chemically synthesized) DNA. A nucleic acid molecule can be double-stranded or single-stranded (i.e., a sense or an antisense single strand). Nucleic acids include, for example, cDNAs encoding the NPs, variant NPs, and chimeric NPs provided herein.

An “isolated nucleic acid” is a nucleic acid that is separated from other nucleic acid molecules that are present in a vertebrate genome, including nucleic acids that normally flank one or both sides of the nucleic acid in a vertebrate genome. The term “isolated” as used herein with respect to nucleic acids also includes any non-naturally-occurring nucleic acid sequence, since such non-naturally-occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences as well as DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not considered an isolated nucleic acid. An “isolated CD-NP nucleic acid,” for example, can be a RNA or DNA molecule containing 9 or more (e.g., 15 or more, 21 or more, 36 or more, or 45 or more) sequential nucleotide bases that encode at least a portion of CD-NP, or a RNA or DNA complementary thereto.

Also provided herein are nucleic acid molecules that can selectively hybridize under stringent hybridization conditions to a nucleic acid molecule encoding a NP (e.g., nucleic acid molecules encoding polypeptides having the amino acid sequences set forth in SEQ ID NO:1, SEQ ID NO:2, and SEQ ID NO:3). The term “selectively hybridize” means to detectably and specifically bind under hybridization and wash conditions that minimize appreciable amounts of detectable binding to nonspecific nucleic acids. For example, high stringency conditions can be used to achieve selective hybridization conditions. Moderate and stringent hybridization conditions include those that are well known in the art. See, for example, sections 9.47-9.51 of Sambrook et al. (1989). As used herein, stringent conditions are those that (1) employ low ionic strength and high temperature for washing, such as 0.015 M NaCl/0.0015 M sodium citrate (SSC) with 0.1% sodium lauryl sulfate (SDS) at 50° C., or (2) employ a denaturing agent such as formamide during hybridization, such as 50% formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C. Alternatively, 50% formamide, 5×SSC (0.75 M NaCl, 0.075 M sodium citrate), 50 mM sodium phosphate (pH 6.8), 0.1% sodium phosphate, 5×Denhardt's solution, sonicated salmon sperm DNA (50 μg/ml), 0.1% sodium dodecylsulfate (SDS), and 10% dextran sulfate at 42° C. can be used, with washes at 42° C. in 0.2×SSC and 0.1% SDS.

Isolated nucleic acid molecules can be produced using standard techniques, including, without limitation, common molecular cloning and chemical nucleic acid synthesis techniques. For example, polymerase chain reaction (PCR) techniques can be used to obtain an isolated nucleic acid containing nucleotide sequence that encodes a NP as provided herein (e.g., a CD-NP, or a variant CD-NP). PCR refers to a procedure or technique in which target nucleic acids are enzymatically amplified. Sequence information from the ends of the region of interest or beyond typically is employed to design oligonucleotide primers that are identical in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. General PCR techniques are described, for example in PCR Primer: A Laboratory Manual, ed. by Dieffenbach and Dveksler, Cold Spring Harbor Laboratory Press, 1995. When using RNA as a source of template, reverse transcriptase can be used to synthesize complementary DNA (cDNA) strands. Ligase chain reaction, strand displacement amplification, self-sustained sequence replication, or nucleic acid sequence-based amplification also can be used to obtain isolated nucleic acids. See, for example, Lewis (1992) Genetic Engineering News 12:1; Guatelli et al. (1990) Proc. Natl. Acad. Sci. USA 87:1874-1878; and Weiss (1991) Science 254:1292.

Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector.

Isolated nucleic acids (e.g., nucleic acids encoding variant NPs) also can be obtained by mutagenesis. For example, a reference sequence can be mutated using standard techniques including oligonucleotide-directed mutagenesis and site-directed mutagenesis through PCR. See, Short Protocols in Molecular Biology, Chapter 8, Green Publishing Associates and John Wiley & Sons, edited by Ausubel et al., 1992. Non-limiting examples of variant NPs art provided herein.

This document also describes exemplary nucleic acid molecules encoding NPs other than ANP, BNP, CNP, DNP, or chimeras or variants thereof. Sources of nucleotide sequences from which nucleic acid molecules encoding a NP, or the nucleic acid complement thereof, can be obtained include total or polyA+RNA from any eukaryotic source, including reptilian (e.g., snake) or mammalian (e.g., human, rat, mouse, canine, bovine, equine, ovine, caprine, or feline) cellular source from which cDNAs can be derived by methods known in the art. Other sources of the nucleic acid molecules provided herein include genomic libraries derived from any eukaryotic cellular source, including mammalian sources as exemplified above.

Nucleic acid molecules encoding native NPs can be identified and isolated using standard methods, e.g., as described by Sambrook et al., Molecular Cloning: A Laboratory Manual, Cold Spring Harbor Laboratory Press, NY (1989). For example, reverse-transcriptase PCR (RT-PCR) can be used to isolate and clone NP cDNAs from isolated RNA that contains RNA sequences of interest (e.g., total RNA isolated from human tissue). Other approaches to identify, isolate and clone NP cDNAs include, for example, screening cDNA libraries.

Vectors containing nucleic acids such as those described herein also are provided. A “vector” is a replicon, such as a plasmid, phage, or cosmid, into which another DNA segment may be inserted so as to bring about the replication of the inserted segment. An “expression vector” is a vector that includes one or more expression control sequences, and an “expression control sequence” is a DNA sequence that controls and regulates the transcription and/or translation of another DNA sequence.

In the expression vectors provided herein, a nucleic acid (e.g., a nucleic acid encoding a NP, such as CD-NP) can be operably linked to one or more expression control sequences. As used herein, “operably linked” means incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 to 500 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence. Expression vectors thus can be useful to produce antibodies as well as other multivalent molecules.

Suitable expression vectors include, without limitation, plasmids and viral vectors derived from, for example, bacteriophage, baculoviruses, tobacco mosaic virus, herpes viruses, cytomegalovirus, retroviruses, vaccinia viruses, adenoviruses, and adeno-associated viruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

An expression vector can include a tag sequence designed to facilitate subsequent manipulation of the expressed nucleic acid sequence (e.g., purification or localization). Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide including at either the carboxyl or amino terminus.

Host cells containing vectors also are provided. The term “host cell” is intended to include prokaryotic and eukaryotic cells into which a recombinant expression vector can be introduced. As used herein, “transformed” and “transfected” encompass the introduction of a nucleic acid molecule (e.g., a vector) into a cell by one of a number of techniques. Although not limited to a particular technique, a number of these techniques are well established within the art. Prokaryotic cells can be transformed with nucleic acids by, for example, electroporation or calcium chloride mediated transformation. Nucleic acids can be transfected into mammalian cells by techniques including, for example, calcium phosphate co-precipitation, DEAE-dextran-mediated transfection, lipofection, electroporation, or microinjection. Suitable methods for transforming and transfecting host cells are found in Sambrook et al., Molecular Cloning: A Laboratory Manual (2^{nd} edition), Cold Spring Harbor Laboratory, New York (1989), and reagents for transformation and/or transfection are commercially available (e.g., LIPOFECTIN® (Invitrogen); FUGENE® (Roche, Indianapolis, Ind.); and SUPERFECT® (Qiagen, Valencia, Calif.)).

### Compositions

The compounds described herein (e.g., chimeric and variant NPs, such as CD-NP), or nucleic acids encoding the polypeptides described herein, can be incorporated into compositions for administration to a subject (e.g., a subject suffering from or at risk for AMI or AHF). Methods for formulating and subsequently administering therapeutic compositions are well known to those in the art. Dosages typically are dependent on the responsiveness of the subject to the compound, with the course of treatment lasting from several days to several months, or until a suitable response is achieved. Persons of ordinary skill in the art routinely determine optimum dosages, dosing methodologies and repetition rates. Optimum dosages can vary depending on the relative potency of an antibody, and generally can be estimated based on the EC₅₀ found to be effective in in vitro and/or in vivo animal models. Compositions containing the compounds (e.g., NPs) and nucleic acids provided herein may be given once or more daily, weekly, monthly, or even less often, or can be administered continuously for a period of time (e.g., hours, days, or weeks). As described herein, for example, a NP or a composition containing a NP can be administered at a dose of at least about 0.01 ng NP/kg to about 100 mg NP/kg of body mass at or about the time of reperfusion, or can be administered continuously as an infusion beginning at or about the time of reperfusion and continuing for one to seven days (e.g., at a dose of about 0.01 ng NP/kg/minute to about 0.5 μg NP/kg/minute).

The NPs and nucleic acids can be admixed, encapsulated, conjugated or otherwise associated with other molecules, molecular structures, or mixtures of compounds such as, for example, liposomes, receptor or cell targeted molecules, or oral, topical or other formulations for assisting in uptake, distribution and/or absorption.

In some embodiments, a composition can contain a NP as provided herein in combination with a pharmaceutically acceptable carrier. Pharmaceutically acceptable carriers include, for example, pharmaceutically acceptable solvents, suspending agents, or any other pharmacologically inert vehicles for delivering antibodies to a subject. Pharmaceutically acceptable carriers can be liquid or solid, and can be selected with the planned manner of administration in mind so as to provide for the desired bulk, consistency, and other pertinent transport and chemical properties, when combined with one or more therapeutic compounds and any other components of a given pharmaceutical composition. Typical pharmaceutically acceptable carriers include, without limitation: water; saline solution; binding agents (e.g., polyvinylpyrrolidone or hydroxypropyl methylcellulose); fillers (e.g., lactose or dextrose and other sugars, gelatin, or calcium sulfate); lubricants (e.g., starch, polyethylene glycol, or sodium acetate); disintegrates (e.g., starch or sodium starch glycolate); and wetting agents (e.g., sodium lauryl sulfate).

Pharmaceutical compositions containing molecules described herein can be administered by a number of methods, depending upon whether local or systemic treatment is desired. Administration can be, for example, parenteral (e.g., by subcutaneous, intrathecal, intraventricular, intramuscular, or intraperitoneal injection, or by intravenous (i.v.) drip); oral; topical (e.g., transdermal, sublingual, ophthalmic, or intranasal); or pulmonary (e.g., by inhalation or insufflation of powders or aerosols), or can occur by a combination of such methods. Administration can be rapid (e.g., by injection) or can occur over a period of time (e.g., by slow infusion or administration of slow release formulations).

Compositions and formulations for parenteral, intrathecal or intraventricular administration include sterile aqueous solutions (e.g., sterile physiological saline), which also can contain buffers, diluents and other suitable additives (e.g., penetration enhancers, carrier compounds and other pharmaceutically acceptable carriers).

Compositions and formulations for oral administration include, for example, powders or granules, suspensions or solutions in water or non-aqueous media, capsules, sachets, or tablets. Such compositions also can incorporate thickeners, flavoring agents, diluents, emulsifiers, dispersing aids, or binders.

Formulations for topical administration include, for example, sterile and non-sterile aqueous solutions, non-aqueous solutions in common solvents such as alcohols, or solutions in liquid or solid oil bases. Such solutions also can contain buffers, diluents and other suitable additives. Pharmaceutical compositions and formulations for topical administration can include transdermal patches, ointments, lotions, creams, gels, drops, suppositories, sprays, liquids, and powders. Conventional pharmaceutical carriers, aqueous, powder or oily bases, thickeners and the like may be useful.

Pharmaceutical compositions include, but are not limited to, solutions, emulsions, aqueous suspensions, and liposome-containing formulations. These compositions can be generated from a variety of components that include, for example, preformed liquids, self-emulsifying solids and self-emulsifying semisolids. Emulsion formulations are particularly useful for oral delivery of therapeutic compositions due to their ease of formulation and efficacy of solubilization, absorption, and bioavailability. Liposomes can be particularly useful due to their specificity and the duration of action they offer from the standpoint of drug delivery.

Compositions provided herein can contain any pharmaceutically acceptable salts, esters, or salts of such esters, or any other compound which, upon administration to a subject, is capable of providing (directly or indirectly) the biologically active metabolite or residue thereof for the relevant compound (e.g., NP). Accordingly, for example, this document describes pharmaceutically acceptable salts of NPs, prodrugs and pharmaceutically acceptable salts of such prodrugs, and other bioequivalents. A prodrug is a therapeutic agent that is prepared in an inactive form and is converted to an active form (i.e., drug) within the body or cells thereof by the action of endogenous enzymes or other chemicals and/or conditions. The term “pharmaceutically acceptable salts” refers to physiologically and pharmaceutically acceptable salts of the NPs useful in methods provided herein (i.e., salts that retain the desired biological activity of the parent NPs without imparting undesired toxicological effects). Examples of pharmaceutically acceptable salts include, but are not limited to, salts formed with cations (e.g., sodium, potassium, calcium, or polyamines such as spermine); acid addition salts formed with inorganic acids (e.g., hydrochloric acid, hydrobromic acid, sulfuric acid, phosphoric acid, or nitric acid); salts formed with organic acids (e.g., acetic acid, citric acid, oxalic acid, palmitic acid, or fumaric acid); and salts formed with elemental anions (e.g., bromine, iodine, or chlorine).

Compositions additionally can contain other adjunct components conventionally found in pharmaceutical compositions. Thus, the compositions also can include compatible, pharmaceutically active materials such as, for example, antipruritics, astringents, local anesthetics or anti-inflammatory agents, or additional materials useful in physically formulating various dosage forms of the compositions, such as dyes, flavoring agents, preservatives, antioxidants, opacifiers, thickening agents, and stabilizers. Furthermore, the composition can be mixed with auxiliary agents, e.g., lubricants, preservatives, stabilizers, wetting agents, emulsifiers, salts for influencing osmotic pressure, buffers, colorings, flavorings, penetration enhancers, and aromatic substances. When added, however, such materials should not unduly interfere with the biological activities of the other components within the compositions.

Pharmaceutical formulations as disclosed herein, which can be presented conveniently in unit dosage form, can be prepared according to conventional techniques well known in the pharmaceutical industry. Such techniques include the step of bringing into association the active ingredients (i.e., the antibodies) with the desired pharmaceutical carrier(s). Typically, the formulations can be prepared by uniformly and intimately bringing the active ingredients into association with liquid carriers or finely divided solid carriers or both, and then, if necessary, shaping the product. Formulations can be sterilized if desired, provided that the method of sterilization does not interfere with the effectiveness of the molecules(s) contained in the formulation.

### Methods for Reducing or Inhibiting Cardiac Remodeling

This document also provides for the use of compounds (e.g., NPs) as disclosed herein for treatment of, for example, AHF and AMI, and to inhibit or reduce cardiac remodeling. Thus, the compounds and nucleic acid molecules provided herein can be administered to a mammal (e.g., a human or a non-human mammal) in order to reduce or inhibit cardiac remodeling that can occur, for example, after MI. In some embodiments, the term “administering” as used herein includes prescribing a compound or composition for use by a mammal in order to reduce or inhibit cardiac remodeling. In some embodiments, for example, a NP or a composition as provided herein can be administered to a mammal diagnosed as having had an AMI. The composition or NP can be administered at any suitable dose, depending on various factors including, without limitation, the agent chosen, the disease, and whether prevention or treatment is to be achieved. Administration can be local or systemic.

In some embodiments, a NP or a composition containing a NP can be administered at a dose of at least about 0.01 ng NP/kg to about 100 mg NP/kg of body mass (e.g., about 10 ng NP/kg to about 50 mg NP/kg, about 20 ng NP/kg to about 10 mg NP/kg, about 0.1 ng NP/kg to about 20 ng NP/kg, about 3 ng NP/kg to about 10 ng NP/kg, or about 50 ng NP/kg to about 100 μg/kg) of body mass, although other dosages also may provide beneficial results. In some cases, a composition containing an NP such as CD-NP or a variant thereof can be administered as a continuous intravenous infusion beginning at or about the time of reperfusion (i.e., at the time the occluded artery is opened), and continuing for one to seven days (e.g., one, two, three, four, five, six, or seven days). Such a composition can be administered at a dose of, for example, about 0.1 ng NP/kg/minute to about 500 ng NP/kg/minute (e.g., about 0.5 ng NP/kg/minute, about 1 ng NP/kg/minute, about 2 ng NP/kg/minute, about 3 ng NP/kg/minute, about 5 ng NP/kg/minute, about 7.5 ng NP/kg/minute, about 10 ng NP/kg/minute, about 12.5 ng NP/kg/minute, about 15 ng NP/kg/minute, about 20 ng NP/kg/minute, about 25 ng NP/kg/minute, about 30 ng NP/kg/minute, about 50 ng NP/kg/minute, about 100 ng NP/kg/minute, or about 300 ng NP/kg/minute). In some embodiments, a composition containing a NP can be administered before reperfusion (e.g., about one hour prior to reperfusion), either as one or more individual doses or as a continuous infusion beginning about one hour prior to reperfusion). For example, a composition can be administered beginning about one hour, about 45 minutes, about 30 minutes, or about 15 minutes prior to reperfusion. In some cases, a composition containing a NP as provided herein can be administered after reperfusion (e.g., within about ten hours of reperfusion), and can be administered either as one or more individual doses or as a continuous infusion beginning within about ten hours of reperfusion. For example, a composition can be administered about one hour, about two hours, about three hours, about four hours, about five hours, about six hours, about seven hours, about eight hours, about nine hours, or about ten hours after reperfusion.

In some embodiments, a NP or a composition containing a NP can be administered via a first route (e.g., intravenously) for a first period of time, and then can be administered via another route (e.g., subcutaneously) for a second period of time. For example, a composition containing a NP can be intravenously administered to a mammal (e.g., a human) at a dose of about 0.1 ng NP/kg/minute to about 300 ng NP/kg/minute (e.g., about 1 ng NP/kg/minute to about 15 ng NP/kg/minute, about 3 ng NP/kg/minute to about 10 ng NP/kg/minute, or about 10 ng NP/kg/minute to about 30 ng NP/kg/minute) for one to seven days (e.g., one, two, three, four, five, six, or seven days), and subsequently can be subcutaneously administered to the mammal at a dose of about 10 ng NP/kg/day to about 100 ng NP/kg/day (e.g., about 10 ng NP/kg/day, about 20 ng NP/kg/day, about 25 ng NP/kg/day, about 30 ng NP/kg/day, about 50 ng NP/kg/day, or about 100 ng NP/kg/day) for five to 30 days (e.g., seven, 10, 14, 18, 21, 24, or 27 days).

The methods provided herein can include administering to a mammal an effective amount of a NP (e.g., a chimeric or variant NP) or a nucleic acid encoding a NP, or an effective amount of a composition containing such a molecule. As used herein, the term “effective amount” is an amount of a molecule or composition that is sufficient to alter one or more (e.g., one, two, three, four, five, six, seven, eight, nine, or ten) parameters indicative of reduced cardiac remodeling and/or kidney protection in a mammalian recipient by at least 10% (e.g., 10%, 15%, 20%, 25%, 30%, 40%, 50%, 60%, 70%, 75%, 80%, 85%, 90%, 95%, 99%, or 100%). For example, an effective amount of a NP as provided herein is an amount that can increase ejection fraction, GFR, UNaV, or UV by at least 10%, and/or that can decrease PRA, LV mass, CF proliferation, PWCP, RAP, MAP, aldosterone levels, LV hypertrophy, ventricular fibrosis, LV end systolic diameter, PFRNa, or DFRNa by at least 10%, and/or that can result in cardiac unloading. In some embodiments, a method can include administering to a mammal an amount of a NP or composition that is sufficient to alter one or more parameters indicative of reduced cardiac remodeling and/or kidney protection by at least 50%.

In some embodiments, for example, an “effective amount” of a NP as provided herein can be an amount that reduces PRA and MAP and increases GFR and UV in a treated mammal by at least 10% as compared to the levels of those parameters in the mammal prior to administration of the NP or without administration of the NP (e.g., the level of the parameters observed in a previous MI episode). Such parameters can be measured using, for example, the methods described in the Examples below.

The invention will be further described in the following examples, which do not limit the scope of the invention described in the claims.

### EXAMPLES

### Example 1

### Materials and Methods

Polypeptide synthesis: Two peptides were synthesized based on the known sequences of DNP and CNP. First, the linear 15-AA C-terminal sequence of DNP (PSLRDPRPNAPSTSA; SEQ ID NO:2; FIG. 1) was synthesized. This peptide was termed “C-terminus.” Second, a chimera of CNP (SEQ ID NO:1; FIG. 1) and the C-terminus of DNP was synthesized (GLSKGCFGLKLDRIGSMSGLGCPSLRDPRPNAPS TSA; SEQ ID NO:3). The 37-AA chimeric peptide was termed “CD-NP.” The two peptides were synthesized using solid phase methods by the Mayo Protein Core Facility on an ABI 431A Peptide Synthesizer (PE Biosystems, Foster City, Calif.) on a preloaded Wang resin with N-Fmoc-L-amino acids (SynPep, Dublin, Calif.). Coupling of each amino acid to the resin-linked peptide was performed in 1-methyl-2-pyrrolidinone (NMP) for 40 minutes. Each Fmoc amino acid was activated in a solution of HOBT/DCC in NMP for 30 minutes. Deprotection of the Fmoc protecting group was performed with 20% piperidine in NMP for 20 minutes before coupling of the next activated amino acid. Subsequently, peptides were deprotected and removed from the resin by treatment with a mixture of 82.5% Trifluoroacetic acid (TFA)/5% water/5% thioanisole/2.5% ethanedithiol/5% phenol for 2 hours at room temperature. Each peptide was washed by precipitation in 3×50 ml of cold methyl t-butyl ether and purified by reverse phase HPLC on a Jupiter C18 column (Phenomenex, Torrance, Calif.) in 0.1% TFA/water with a gradient of 10-70% B in 50 minutes.

The identity of each peptide was verified by electrospray ionization (ESI) mass analysis on a Perkin/Elmer Sciex API 165 Mass Spectrometer (PE Biosystems, Foster City, Calif.). Disulfide bridges in CD-NP were formed by overnight air oxidation in a 50 mM ammonium bicarbonate pH 8.5 buffer.

Integrative In Vivo Biological Actions of C-Terminus and CD-NP in Normal Canines: Experiments were performed in five separate groups of normal anesthetized dogs. All studies conformed to the guidelines of the American Physiological Society and were approved by the Mayo Clinic Animal Care and Use Committee.

On the evening before the experiments, 300 mg of lithium carbonate was administered orally for the assessment of segmental renal tubular function, and then the animals were fasted overnight. On the day of the acute experiment, all dogs were anesthetized with pentobarbital sodium given intravenously (30 mg/kg). Dogs were mechanically ventilated (Harvard respirator, Harvard Apparatus, Millis, Mass.) with 4 L/minute of supplemental oxygen. A left lateral flank incision was made and the left kidney was exposed. The ureter was cannulated for timed urine collection, and a calibrated electromagnetic flow probe was placed around the left renal artery and connected to a flowmeter (model FM 5010, King, N.C.) for monitoring renal blood flow (RBF). Finally, the right femoral vein was cannulated with two polyethylene catheters (PE-240), one for infusion of inulin and the other for the infusion of C-terminus. The right femoral artery was cannulated for direct arterial blood pressure measurement and arterial blood sampling. For determination of cardiac filling pressures in the groups that received CD-NP or BNP, and for measurement of cardiac output, a Swan-Ganz catheter (Edwards, Mountain View, Calif.) was inserted into the right internal jugular vein.

After completion of the surgical preparation, a priming dose of inulin (ICN Biomedicals, Cleveland, Ohio) was injected, followed by a constant infusion of 1 ml/minute. The dogs were allowed to equilibrate for 60 minutes without intervention. After the equilibration period, a 30 minute baseline clearance (Baseline) was performed. This was followed by a 15 minute lead-in period during which C-terminus infusion at 42 ng/kg/min in the first group (n=6) was begun intravenously, and after which a second 30 minute clearance (C-terminus) period was performed. A time control with intravenous saline at 1 ml/min (n=6) was performed to serve as a control for the CD-NP group. During each clearance, renal hemodynamic and excretory response was recorded. In the third group, CD-NP was infused at three concentrations (10, 50 and 100 ng/kg/min) for 30 minutes, with a 15-minute lead in period for each dose. Groups 4 (n=6) and 5 (n=6 received an equimolar concentration of 10 and 50 ng/kg/min of CD-NP or BNP, respectively.

Plasma and urine electrolytes, including lithium, were measured by flame-emission spectrophotometer (IL943, Flame Photometer, Instrumentation Laboratory, Lexington, Mass.). Plasma and urine inulin concentrations were measured by the anthrone method, and GFR was measured by the clearance of inulin. The lithium clearance technique was employed to estimate the PFRNa and DFRNa. PFRNa was calculated by the following formula: [1−(lithium clearance/GFR)]×100. DFRNa was calculated by the formula: [(lithium clearance−sodium clearance)/lithium clearance]×100. Plasma and urinary cGMP and plasma renin activity (PRA) were measured using commercially available radioimmunoassay as described previously.

In Vitro Studies on CD-NP cGMP Generation and Inhibition of Cell Proliferation in Human Cardiac Fibroblasts: Studies were performed in human CFs (ScienCell, San Diego, Calif.) in accordance with methods previously described (Tsuruda et al. (2002) Circ Res 91:1127-1134). Only cell passages 1 through 4 were used for experiments. Cells were exposed to CD-NP (10⁻¹¹ to 10⁻⁶ M) for 15 minutes to determine cGMP activation. The samples were assayed using a competitive RIA cGMP kit (Perkin-Elmer, Boston, Mass.). Briefly, samples and standards were incubated with anti-human cGMP polyclonal antibody and I¹²⁵-antigen for 18 hours. Cyclic GMP assay buffer was added to the samples and they were centrifuged for 20 minutes at 2500 rpm. The free fraction was aspirated off and the bound fraction was counted and concentrations determined. Values are expressed as pmol/ml. There was no cross-reactivity with ANP, BNP, CNP, or ET, and <0.001% cross-reactivity with cAMP, GMP, GDP, ATP, GTP.

For CF proliferation studies, 70-80% confluent cells at passage 1-4 cells were treated for 24 hours with 10⁻⁸ M Cardiotrophin-1 to induce cell proliferation. CD-NP at a concentration of 10⁻⁸ M was added to Cardiotrophin-1-stimulated CFs to determine its effect on cell proliferation. Untreated fibroblasts were processed as a control. Colormetric bromodeoxyuridine (BrdU) cell proliferation ELISA (Roche, Indianapolis, Ind.) was performed as directed. Briefly, CFs were labeled with BrdU for 2 hours in a CO₂ 37° C. incubator. Anti-BrdU was added and allowed to react for 90 minutes at room temperature. The anti-BrdU was removed and the CFs were washed three times with a washing solution. Colormetric substrate solution was added and color was allowed to develop for 30 minutes. Absorbance at 370 nm was measured on a SpectraMax spectrophotometer (Molecular Devices, Sunnyvale, Calif.).

Statistical Analysis: Results of quantitative studies are expressed as mean±standard error. Statistical comparison within one group when comparing two clearances was performed using Student's t test, and when comparing multiple clearances, repeated measures ANOVA followed by post hoc Dunnett's test was used. Statistical comparison between groups was performed using Two-way ANOVA followed by Bonferroni post-test. Statistical significance was accepted for P value<0.05.

### Example 2

### In Vivo Actions of C-Terminus of DNP and CD-NP

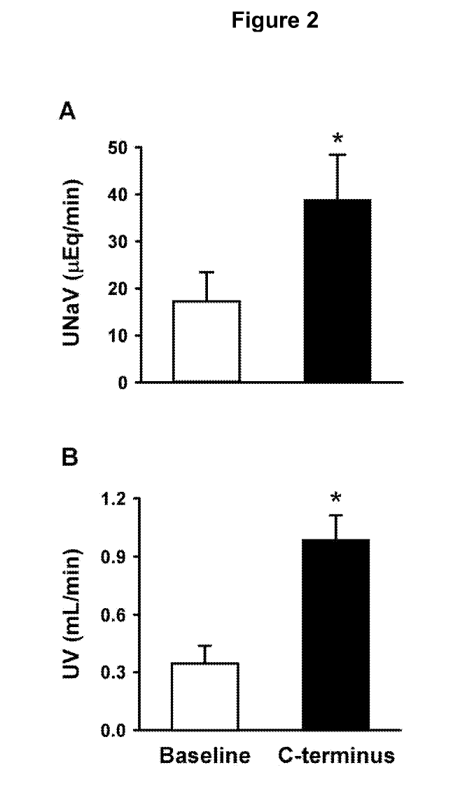

The C-terminus of DNP that is lacking the core ring structure of DNP was natriuretic and diuretic (FIG. 2). The natriuretic action was localized to the proximal segment of the renal nephron, as a decrease in proximal fractional sodium reabsorption (PFRNa) was observed, suggesting that this part of the nephron contributes to the renal actions of this small peptide (Table 2). There was no notable change in glomerular filtration rate (GFR), renal blood flow (RBF) or urinary cGMP excretion. Importantly, in a time control group that also received saline at 1 ml/minute as in the C-terminus group, parameters of renal function were unchanged.

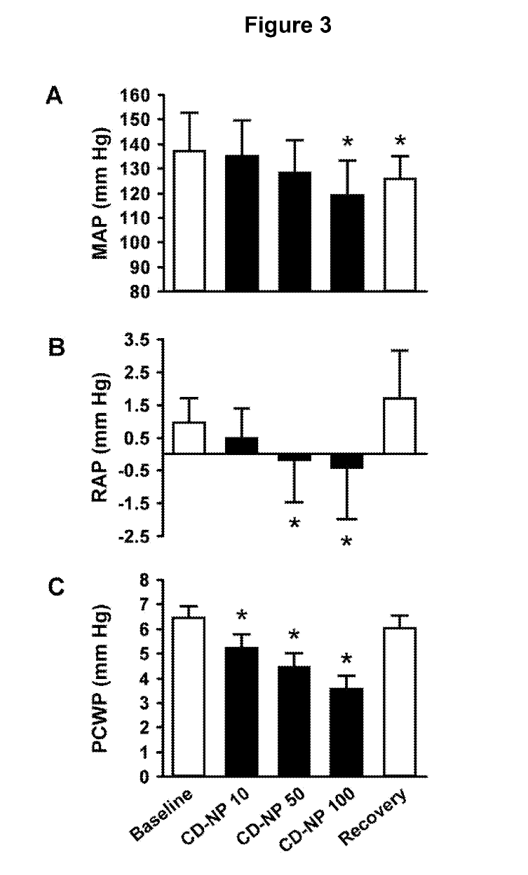

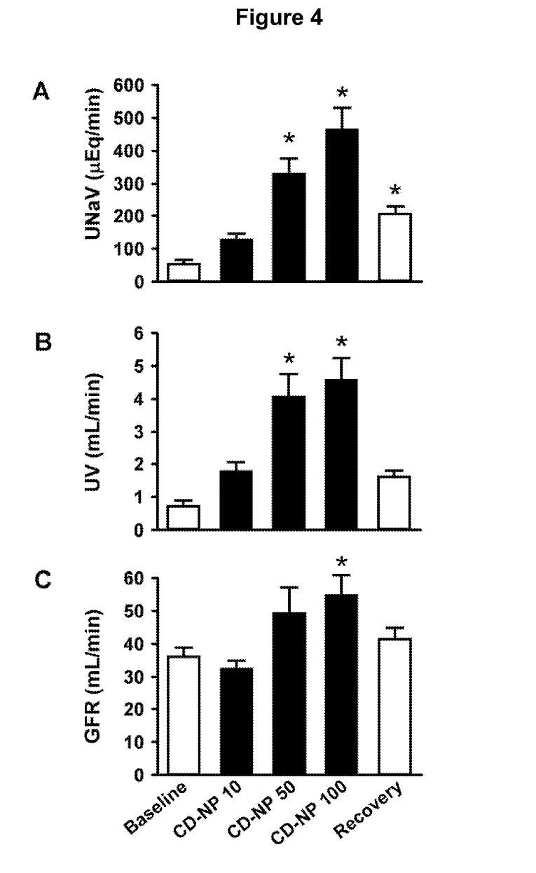

FIG. 3 reports the in vivo responses to CD-NP infusion. All three doses of CD-NP decreased pulmonary capillary wedge pressure (PCWP), while two doses decreased right atrial pressure (RAP). These cardiac unloading responses were associated with minimal decreases in mean arterial pressure (MAP) even during the infusion of high dose of CD-NP. Potent natriuretic and diuretic responses with CD-NP also were observed in normal canines (FIG. 4). Specifically, both medium and high dose CD-NP increased urinary excretion of sodium (UNaV) and urine flow (UV) (FIG. 4). Despite a slight decrease in MAP during the infusion of high dose of CD-NP, GFR increased.

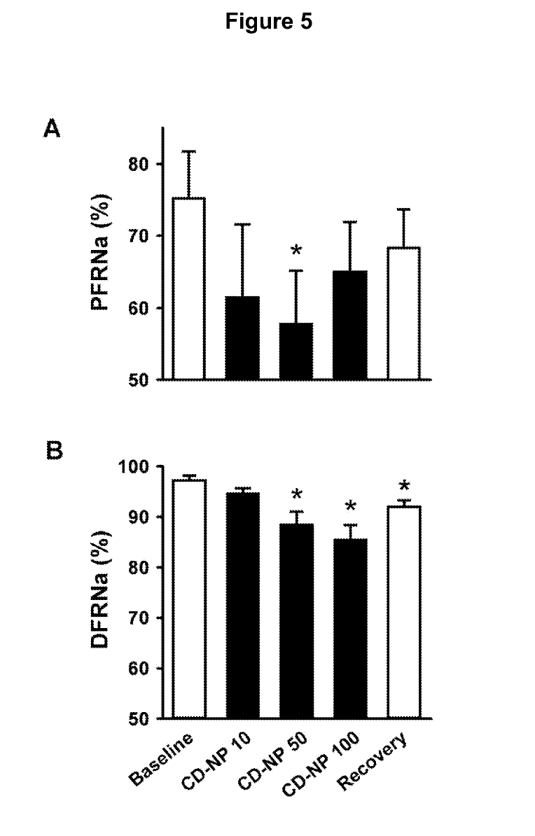

Natriuresis and diuresis involved a decrease in proximal tubular reabsorption of sodium, as PFRNa during the infusion of medium dose of CD-NP was reduced. In addition, a decrease in distal fractional sodium reabsorption (DFRNa) during the infusion of medium and high dose of CD-NP was observed, indicating that CD-NP also involves activities in the terminal nephron (FIG. 5). These renal parameters returned to baseline levels after discontinuation of CD-NP infusion. These actions also were associated with suppression of plasma renin activity (PRA) during the infusion of low and medium doses of CD-NP (Table 3). PRA returned to baseline levels after discontinuation of infusion. Medium and high doses of CD-NP increased plasma and urinary cGMP (Table 3).

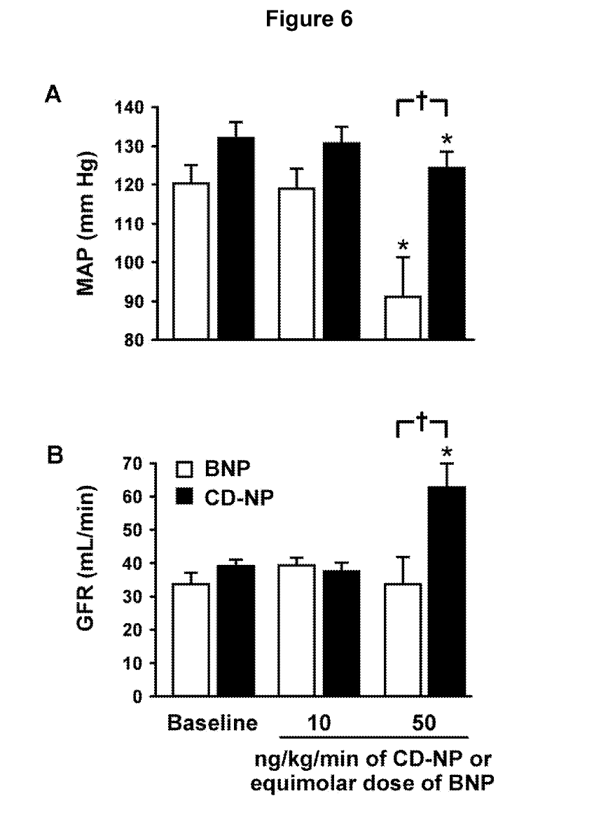

CD-NP significantly increased GFR at a dose of 50 ng/kg/minute, in contrast to a lack of increase in GFR in the group receiving an equimolar dose of BNP (FIG. 6). Importantly, this dose of CD-NP was associated with a less hypotensive action than BNP.

TABLE 2
Renal hemodynamic and excretory response to infusion of C-terminus

| | Baseline | C-terminus |
|---|---|---|
| GFR (ml/minute) | 34 ± 6 | 44 ± 6 |
| RBF (ml/minute) | 213 ± 19 | 198 ± 27 |
| PFRNa (%) | 77.9 ± 2.7 | 71.0 ± 2.6* |
| DFRNa (%) | 98.7 ± 0.3 | 97.8 ± 0.5 |
| UcGMPV (pmol/minute) | 1674 ± 213 | 2275 ± 231 |

Data are expressed as means ± SE;
C-terminus, infusion of 42 ng/kg/minute of C-terminus of DNP (n = 6);
GRF, glomerular filtration rate;
RBF, renal blood flow;
PFRNa, proximal fractional sodium reabsorption;
DFRNa, distal fractional sodium reabsorption;
UcGMPV, urinary cGMP excretion;
*P < 0.05 vs. baseline.

TABLE 3
Neurohumoral response to infusion of CD-NP in normals

| | Baseline | CD-NP 10 | CD-NP 50 | CD-NP 100 | Recovery |
|---|---|---|---|---|---|
| PcGMP (pmol/ml) | 9.4 ± 1.2 | 14.6 ± 1.6 | 30.3 ± 2.1* | 50.1 ± 2.1* | 32.7 ± 6.7* |
| UcGMPV (pmol/minute) | 1648 ± 151 | 2133 ± 234 | 4566 ± 621* | 9999 ± 1590* | 6661 ± 734* |
| PRA (ng/ml/hour) | 8.2 ± 0.8 | 5.3 ± 1.1* | 3.9 ± 0.7* | 6.3 ± 1.1 | 9.0 ± 1.8 |

Data are expressed as means ± SE;
CD-NP 10, infusion of 10 ng/kg/minute of CD-NP;
CD-NP 50, infusion of 50 ng/kg/minute of CD-NP;
CD-NP 100, infusion of 100 ng/kg/minute of CD-NP (n = 8 except n = 6 for high dose);
PcGMP, plasma cGMP;
UcGMPV, urinary cGMP excretion;
PRA, plasma renin activity;
*P < 0.05 vs. baseline.

### Example 3

### CD-NP Mediated cGMP Generation in Cardiac Fibroblasts and Inhibition of Proliferation

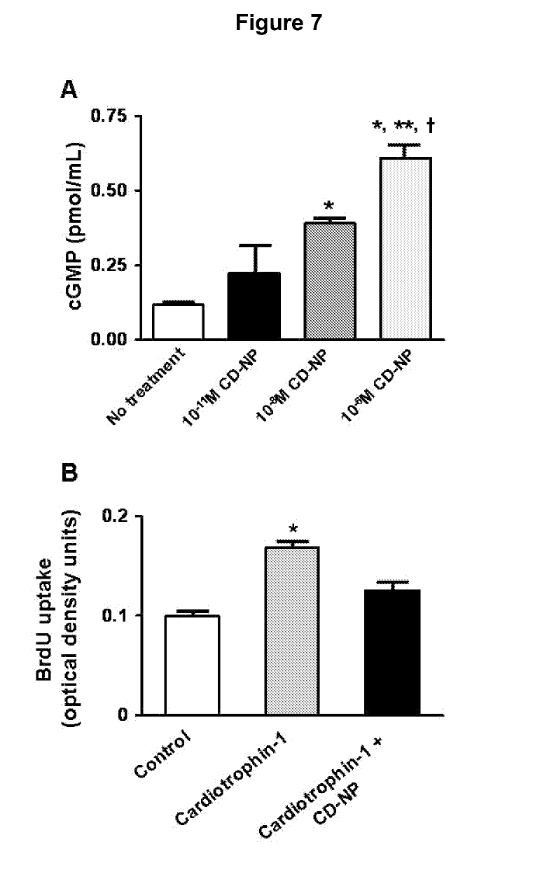

In vitro studies in cultured human CFs demonstrated that CD-NP activated cGMP in a dose dependent manner (FIG. 7A). Further, when compared to CNP, BNP and DNP, the cGMP generated with CD-NP was similar to CNP and significantly greater (p<0.05) than DNP and BNP at 10⁻⁶ M. Experiments were then conducted to determine whether CD-NP possessed anti-proliferative actions, using the pro-fibrotic and hypertrophic cytokine, Cardiotrophin-1. This cytokine is a markedly robust activator of CFs and is activated in states such as AMI and heart failure (Jougasaki et al. (2000) Circulation 101:14-17; Talwar et al. (2002) Clin Sci (Lond) 102:9-14; and Tsuruda et al. (2002) Circ Res 90:128-134). CD-NP suppressed cell proliferation in human CFs induced by Cardiotrophin-1, as assessed by BrdU uptake as a measure of DNA synthesis and cellular proliferation (FIG. 7B).

### Example 4

### In Vivo Studies to Assess CD-NP Effects on LV Mass in Rats After MI

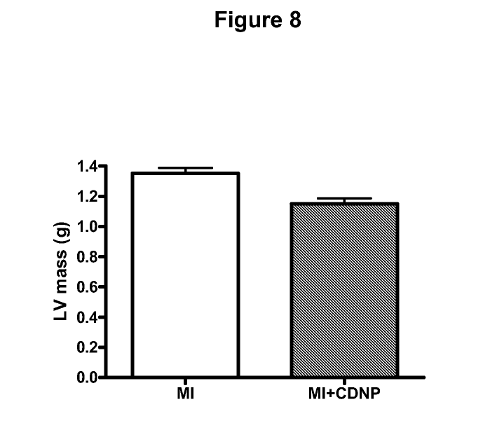

Myocardial infarction (MI) was produced by left anterior descending coronary artery ligation in Wistar rats (150-250 g), and an osmotic mini pump (Alzet Osmotic pump model 2ML2) was inserted in the back of each rat in the subcutaneous space. CD-NP was administered subcutaneously at a dose of 1.7×10⁻⁷ g/kg/minute for 2 weeks. LV Mass was assessed by echocardiogram before the acute experiment and 3 weeks after MI. Treatment with CD-NP resulted in decreased LV mass after 3 weeks of MI as compared to the control MI-no treatment group (FIG. 8). Specifically, LV mass in the MI group was 1.351±0.03764 (N=10), while LV mass in the MI+CD-NP group was 1.150±0.03651 (N=6; p=0.0031).

### Example 5

### In Vivo Actions of CD-NP and CNP-C

Studies in animals were performed in male mongrel dogs (n=25) in accordance with the National Institutes of Health Guide for the Care and Use of Laboratory Animals. The evening before the experiment, each dog was fasted with ad lib access to water and was given lithium carbonate 300 mg orally for assessment of renal tubular function the next day. Canines were anesthetized with pentobarbital sodium (induction 6-20 mg/kg i.v., maintenance 5-15 mg/kg/h i.v.) and fentanyl (0.04-0.12 mg/kg i.v., maintenance 0.04-0.18 mg/kg/h), and were intubated and mechanically ventilated (Harvard Apparatus, Holliston, Mass.) with 5 L/min of O₂ (tidal volume 15 mL/kg, 12 cycles/min). The femoral artery was cannulated for blood pressure monitoring and for blood sampling, and the femoral vein was cannulated for infusion of inulin and normal saline. The saphenous vein was cannulated for peptide infusion. A balloon-tipped thermodilution catheter (Edwards Lifesciences, Irvine, Calif.) was used for monitoring hemodynamics while the left kidney was exposed via a flank incision, and the ureter was cannulated for timed urine collection. An electromagnetic flow probe was placed on the renal artery for measuring renal blood flow (Burnett et al. (1984) Am J Physiol 247:F863-866). A weight-adjusted bolus of inulin was given, followed by an infusion (1 mL/min) to achieve plasma levels of 40 to 60 mg/dL (Burnett et al. (1984) supra; Chen et al. (2005) Am J Physiol Regul Integr Comp Physiol 288:R1093-1097; and Margulies et al. (1991) J Clin Invest 88:1636-1642) for measuring GFR by inulin clearance.

Following a 60 minute equilibration period, a 30 minute pre-infusion clearance was obtained. This was followed by a 75 minute continuous infusion of CD-NP (50 ng/kg/min) i.v. (n=10) or an equimolar concentration of CNP (n=9). The continuous infusion period consisted of a 15 minute lead-in and two 30 minute clearances followed by a 30 minute washout and a post-infusion clearance. In 3 additional dogs, the decoy protein, CNP-C (50 ng/kg/min) was tested as a continuous i.v. infusion for 75 minutes. Data are reported for three 30 minute clearances (pre-infusion, at 30 minutes of infusion, and at 60 minutes of infusion).

Plasma and urinary cGMP levels were measured by radioimmunoassays (RIA; Steiner et al. (1972) J Biol Chem 247:1106-1113). Plasma renin activity (Haber et al. (1969) J Clin Endocrinol Metab 29:1349-1355), angiotensin II (Luchner et al. (1996) Hypertension 28:472-477), and aldosterone (Sancho and Haber (1978) J Clin Endocrinol Metab 47:391-396) were quantified. Plasma and urinary lithium levels were measured by flame emission spectrophotometry (model 357, Instrumentation Laboratory, Wilmington, Mass.) for assessment of tubular handling of sodium (Steiner et al., supra).

For assessment of cGMP activation in isolated glomeruli, canines (n=3) were euthanized with pentobarbital sodium and the kidneys were immediately harvested and glomeruli were isolated as previously described (Supaporn et al. (1996) Kidney Int 50:1718-1725), which was based on the method of Chaumet-Riffaud et al. ((1981) Am J Physiol 241:F517-524) with minor modifications. For quantification of cGMP response to the study peptides, aliquots of glomeruli (300 μL, suspended in Krebs buffer) were incubated with CD-NP or CNP (final concentration 10⁻⁵ M) for 10 minutes at 37° C. (following an initial 10 minutes of pre-incubation) in the presence of isobutylmethyl-xanthine (0.3 mM) in a final volume of 500 μL. The controls consisted of the same composition, with the exception that Krebs buffer was used instead of glomeruli suspended in Krebs buffer. The reaction was terminated by the addition of 300 μL of ice-cold trichloroacetic acid (TCA, final conc. 6.6%) and the incubate was centrifuged. An 800 μL supernate aliquot was extracted with ether for cGMP assay (Steiner et al., supra; and Supaporn et al., supra), and the remaining supernate was neutralized with 1N NaOH and analyzed in a protein assay (BCA protein assay, Pierce Biotechnology, Rockford, Ill.). The above procedure was repeated with isolated glomeruli that were pretreated with an NPR-A antagonist, A71915 10 (final concentration 1 μM). Results were corrected for protein levels and are expressed in fmol/μg.

CD-NP (Clinalfa, Läufelfingen, Switzerland) and CNP (Phoenix Pharmaceuticals, Inc., Belmont, Calif., USA) (FIG. 1) were reconstituted in normal saline. Intravenously administered CD-NP (50.0 ng/kg/min; or 13.35 pmol/kg/min), was tested against an equimolar dose of CNP (29.3 ng/kg/min) as a 75 minute continuous infusion. The decoy protein, CNP-C, was custom synthesized (FIG. 1).

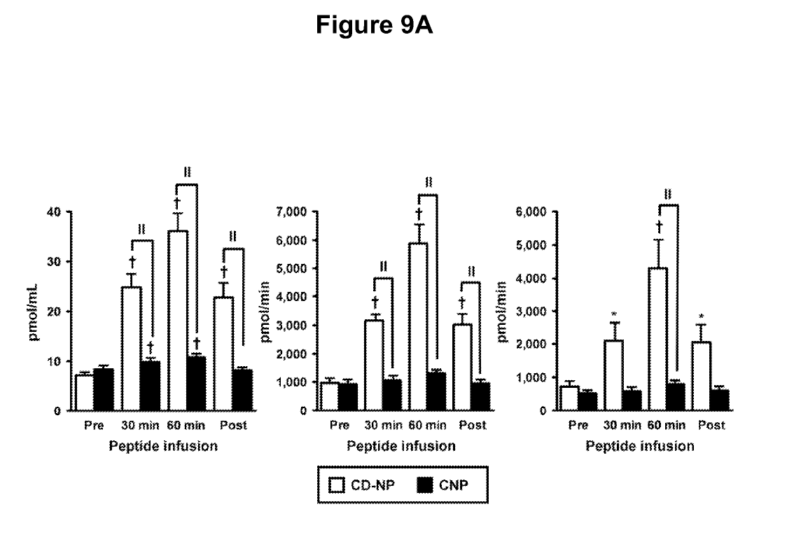

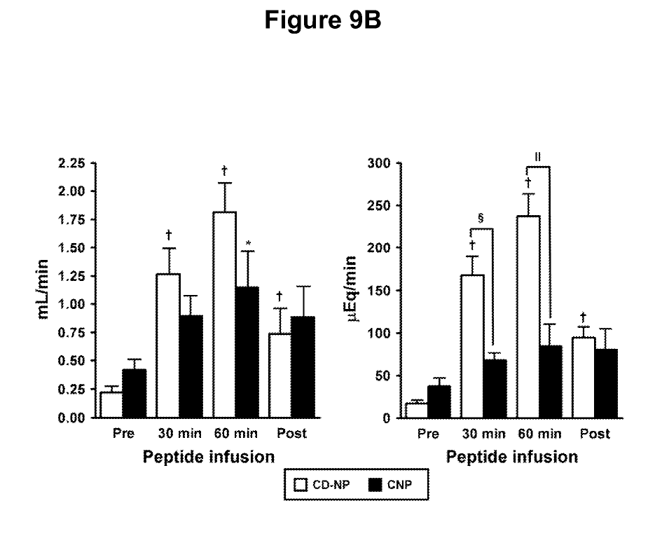

These in vivo studies demonstrated marked activation by CD-NP of the natriuretic peptide second messenger cGMP as revealed by greater increases in plasma and urinary excretion, as well as in net renal generation by the kidney itself, which was distinctly different that the responses to CNP (FIG. 9A). Greater natriuresis, which localized to both proximal and distal tubules as demonstrated by the reductions in proximal and distal fractional reabsorption of sodium (Table 4), and enhanced GFR were observed with CD-NP but not with CNP (FIG. 9B). No significant change in mean arterial pressure was detected in either group (Table 5). Right atrial pressure and pulmonary capillary wedge pressure were reduced by CD-NP, but not by CNP (Table 5). CD-NP significantly suppressed PRA and angiotensin II (ANG II) levels, whereas the changes in the CNP group did not achieve statistical significance (Table 6). Thus, fusion of the C-terminus of DNP to mature CNP transformed CNP into a renal acting, cardiac unloading peptide that suppressed the renin-angiotensin system (RAS) without reducing arterial pressure.

To determine whether the C-terminus of DNP is a strict requirement for in vivo renal and RAS modulating actions a transformed CNP was designed that included a fusion of the amino acid sequence of the N-terminus of CNP into the vacant C-terminus position of CNP. This polypeptide, referred to as CNP-C, consisted of the full-length 22-amino acid CNP and a duplicate of the N-terminal five amino acids fused to the CNP C-terminus (GLSKGCFGLKLDRIGSMSGLGCGKSLG; SEQ ID NO:4). Infusion of this polypeptide did not result in any significant changes in plasma cGMP, urinary cGMP excretion, diuresis, natriuresis, GFR, PRA, or ANG II. Pulmonary capillary wedge pressure (4.3±0.8 to 3.4±0.8 mmHg) and mean arterial pressure (132±6 to 128±5 mmHg) decreased minimally at 60 minutes of infusion versus baseline (P<0.05). Thus, the C-terminus of DNP is specific for transforming CNP into a renal acting and RAS-modulating peptide, as CNP-C failed to augment renal function or suppress RAS. These data provide insights into possible future CNP transformations that could involve fusion of CNP with non-CNP peptide sequences.

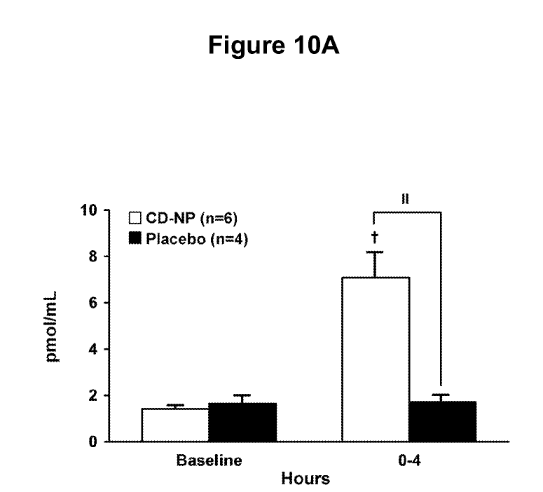

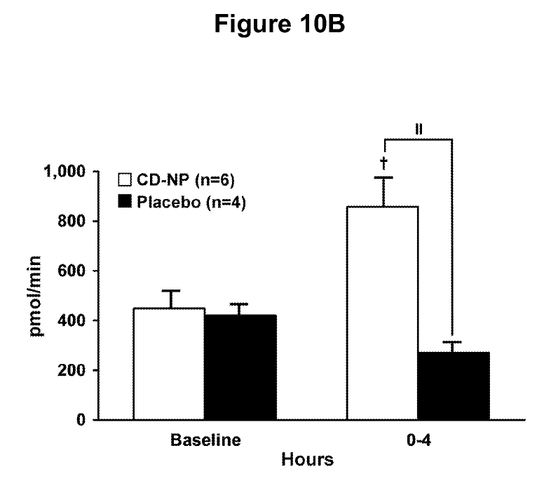

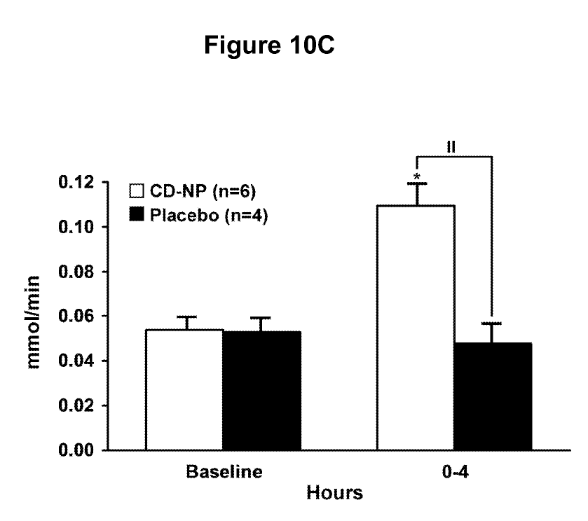

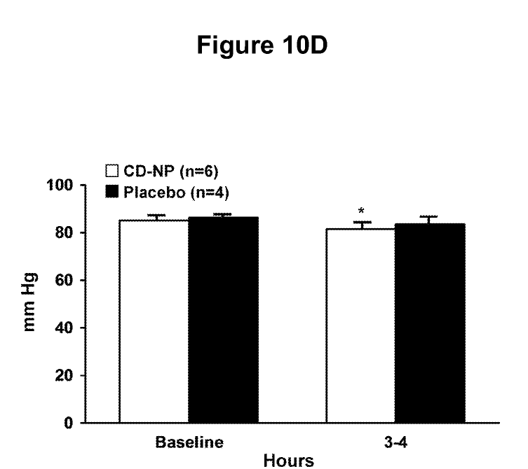

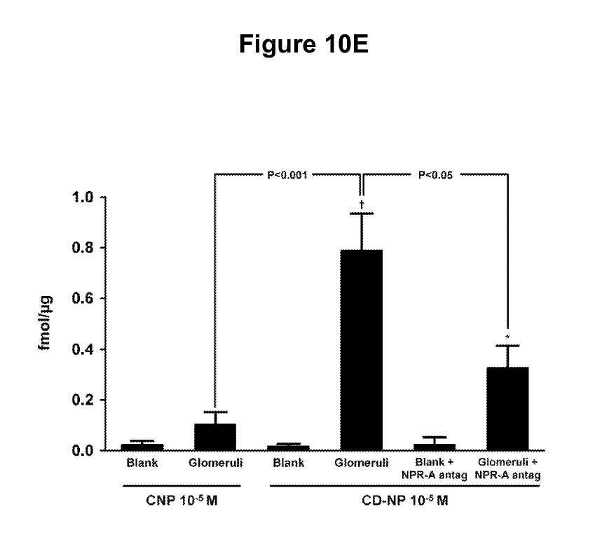

While NPR-B receptors are in the kidney, they have not been associated with mediating actions of glomerular filtration rate or sodium excretion. Rather, such renal actions associated with natriuretic peptides are linked to the NPR-A receptor. Glomeruli were isolated from canines to determine whether the renal actions of CD-NP involve NPR-A, which is the receptor for ANP, BNP, and DNP, considering that the N-terminus of DNP fused to CNP would uniquely activate cGMP in glomeruli that could be abrogated by pharmacological antagonism with a NPR-A antagonist. In isolated canine glomeruli, CD-NP (10⁻⁵ M) activated cGMP to a greater extent than control placebo. At an equimolar concentration (10⁻⁵ M), CD-NP activated cGMP to a greater extent than CNP (FIG. 10E). Pretreatment with of an NPR-A antagonist (Sancho and Haber, supra) (1 μM) resulted in an attenuated cGMP response, demonstrating that CD-NP in the kidney also involves NPR-A activation.

### Example 6

### Human Clinical Trial Results

Following toxicological studies in canines and rodents that confirmed the safety of CD-NP, a first-in-human clinical trial was undertaken in normal human volunteers.

Study protocols: The human clinical trial on CD-NP was conducted in accordance to the Declaration of Helsinki and its amendments, the U.S. Food and Drug Administration Principles of Good Clinical Practice, and International Conference on Harmonization guidelines, where applicable. The clinical trial consisted of 2 stages: an open-label sequential dose escalation study (stage 1) and a randomized, double-blind, placebo (PLB)-controlled study (stage 2). For stage 1, three cohorts of 4 subjects each were enrolled in the dose-escalation study (10, 17.5, or 25 ng/kg/min i.v. for 4 hours). For stage 2, ten subjects were randomized in the double-blind study (6:4 for CD-NP vs. placebo, PLB) which evaluated the maximum tolerated dose (MTD, as determined in stage 1) of CD-NP vs. PLB i.v. for 4 hours.

Main inclusion criteria included: 1) healthy male, post menopausal female, or surgically sterilized female 18 to 60 years of age; 2) BMI within the range of 18 to 34 kg/m2; 3) ability to communicate effectively; 4) no significant disease or abnormal laboratory; 5) normal 12-lead electrocardiogram; 6) being a nonsmokers defined as not having smoked in the past 6 months; and 7) adequately informed of the nature and risks of the study and gave written informed consent prior to receiving study medication.

Main exclusion criteria included: 1) known hypersensitivity or allergy to CD-NP or its components, nesiritide, other natriuretic peptides, or related compounds; 2) women who were pregnant or breast-feeding; 3) any disease or condition (medical or surgical) which, in the opinion of the investigator, might compromise the hematologic, cardiovascular, pulmonary, renal, gastrointestinal, hepatic, or central nervous system; or other conditions that might interfere with the absorption, distribution, metabolism or excretion of study drug, or would place the subject at increased risk; 4) the presence of abnormal laboratory values which were considered clinically significant; 5) positive screen for hepatitis B (HbsAg, hepatitis B surface antigen), hepatitis C (anti HCV, Hepatitis C Antibody), or HIV (anti-HIV 1/2); 6) having received an investigational drug within a period of 30 days prior to enrollment in the study; 6) received any drug therapy within 1 week, or 5 half-lives, prior to administration of the first dose of any study-related treatment. This exclusion was extended to 4 weeks for any drugs known to induce or inhibit hepatic drug metabolism. Use of non-steroidal anti-inflammatory drugs, sulfonamides, probenecid or other drugs known to alter renal or tubular function were specifically prohibited for at least 5 half-lives prior to the first dose of any study related treatment; 7) consumption of alcohol within 48 hours prior to dose administration or during any in patient period; 8) a positive urine drug screen including ethanol, cocaine, tetrahydrocannabinol (THC), barbiturates, amphetamines, benzodiazepines, and opiates; 9) history (within the last 2 years) of alcohol abuse, illicit drug use, significant mental illness, physical dependence to any opioid, or any history of drug abuse or addiction; 10) history of difficulty with donating blood; and 11) having donated blood or blood products within 45 days prior to enrollment.

Data Analysis: Renal, neurohumoral, and hemodynamic data are expressed as mean±SEM. Clearances from minutes 16-45 and from minutes 46-75 following initiation of peptide infusion are denoted by “30 min” and “60 min,” respectively. Within each group, parameters at 30 and 60 minutes of peptide infusion, and post-infusion were compared to pre-infusion values by repeated measures one-way analysis of variance (ANOVA), followed by post-hoc Dunnett's multiple comparison test, where applicable (Chen et al., supra; and Cataliotti et al. (2004) Circulation 109:1680-1685). Comparisons between groups were made by two-way ANOVA followed by Bonferroni post-test (Chen et al., supra; and Cataliotti et al., supra). Statistical significance was defined as P<0.05. GraphPad Prism 4 (GraphPad Software, San Diego, Calif.) was used for statistical analysis of canine data. Statistical analyses for the first-in-human clinical trial were performed using Statistical Analysis Software (version 9). Renal, hemodynamic and neurohumoral data were analyzed for the CD-NP group and the placebo (PLB) group in the randomized, double-blind phase of the clinical trial. Both within group (comparing end of infusion vs. baseline) and between group comparisons (at baseline and at the end of infusion) were made, using parametric and non-parametric tests, as presented in Table 7.

Results: After determining the maximal tolerated dose, CD-NP (17.5 ng/kg/min) was administered for 4 hours and compared to placebo. CD-NP increased plasma cGMP, urinary cGMP excretion, and urinary sodium excretion (FIGS. 10A-10C and Table 7). Urine flow increased in the CD-NP group vs. baseline (1.1±0.2 to 2.3±0.4 mL/min; PLB 1.3±0.2 to 1.6±0.4 mL/min). Mean arterial pressure (FIG. 10D) and GFR did not differ between groups. CD-NP suppressed plasma aldosterone from 21.9±2.7 to 9.5±3.2 ng/dL (P<0.001).

These data demonstrated that the venodilator CNP can be transformed to a renal-enhancing, RAS-suppressing, and cardiac-unloading peptide with minimal effects on blood pressure, making it an attractive next generation therapeutic that could prove useful in AHF. Two principles also are provided by these studies: (1) that the addition of a C-terminus amino acid sequence to the vacant C-terminus of CNP must be specific, as the fusion of an amino acid sequence other that the C-terminus of DNP (i.e., the N-terminus of CNP) lacked an effect; and (2) addition of the C-terminus of DNP transforms CNP into more than an activator of NPR-B, as the polypeptide activated NPR-A in the kidney. Further, these data extend the animal and in vivo investigations to a first-in-human study, establishing the concept that CD-NP activates the cGMP pathway, enhances sodium excretion, and suppresses aldosterone in humans with minimal blood pressure effects compared to the endogenous native peptide CNP.

This study also indicates that CNP can be transformed into a potential therapeutic peptide that, unlike CNP, is natriuretic and aldosterone-suppressing and, like CNP, has minimal hypotensive properties while unloading the heart. The use of a decoy with the N-terminus of CNP provides insight into future chimeric design and underscores the specificity for augmenting the C-terminus of CNP that results in NPR-A agonism in the kidney. In summary, the fusion of the C-terminus of DNP to the full-length 22-AA peptide CNP transforms CNP into a cardiac-unloading, renin-inhibiting and renal-enhancing designer peptide that is relevant to the therapeutics of cardiorenal disease syndromes.

TABLE 4
Renal hemodynamic and excretory response to
intravenous infusion of CD-NP or CNP

| | | | |
|---|---|---|---|
| Pre-I | 30 min I | 60 min I | Post-I |

CD-NP Group (n = 9-10)

| | | | | |
|---|---|---|---|---|
| GFR, | 36.8 ± 1.7^{‡} | 47.5 ± 3.2^{†} | 50.6 ± 3.3^{†} | 53.3 ± 3.5^{†} |
| mL/min | | | | |
| RBF, | 228.5 ± 29.2 | 243.8 ± 27.8 | 265.3 ± 27.1 | 260.9 ± 32.8 |
| mL/min | | | | |
| RVR, × 10⁻³ | 0.76 ± 0.14 | 0.71 ± 0.11 | 0.63 ± 0.10 | 0.71 ± 0.15 |
| mmHg · | | | | |
| L⁻¹ · min | | | | |
| PFR_{Na}, % | 75 ± 2 | 63 ± 2^{†‡} | 57 ± 3^{†∥} | 69 ± 3 |
| DFR_{Na}, % | 98 ± 0.2 | 92 ± 3^{†‡} | 92 ± 1^{†‡} | 96 ± 0.5 |

CNP Group (n = 7-8)

| | | | | |
|---|---|---|---|---|
| GFR, | 52.2 ± 5.3 | 52.7 ± 6.7 | 49.5 ± 4.2 | 48.8 ± 5.6 |
| mL/min | | | | |
| RBF, | 273.4 ± 25.5 | 294.2 ± 30.8 | 277.8 ± 29.2 | 272.0 ± 24.5 |
| mL/min | | | | |
| RVR, × 10⁻³ | 0.49 ± 0.07 | 0.47 ± 0.06 | 0.49 ± 0.06 | 0.49 ± 0.06 |
| mmHg · | | | | |
| L⁻¹ · min | | | | |
| PFR_{Na}, % | 80 ± 3 | 73 ± 2 | 72 ± 1 | 73 ± 2 |
| DFR_{Na}, % | 98 ± 0.6 | 97 ± 0.4 | 97 ± 0.9 | 97 ± 0.8 |

Means ± SEM.
Comparisons within group vs. pre-infusion, pre-I (mean ± SE, P < 0.01^{†}) and between groups (P < 0.05^{‡}, <0.001^{∥}).
GFR, glomerular filtration rate;
RVR, renal vascular resistance;
I, infusion;
PFR_{Na}, proximal fractional reabsorption of Na⁺;
DFR_{Na}, distal fractional reabsorption of Na⁺.

TABLE 5
Cardiovascular hemodynamic response to
intravenous infusion of CD-NP or CNP

| | | | |
|---|---|---|---|
| Pre-I | 30 min I | 60 min I | Post-I |

CD-NP Group (n = 9-10)

| | | | | |
|---|---|---|---|---|
| MAP, mmHg | 127 ± 4 | 124 ± 5 | 122 ± 6 | 126 ± 7 |
| PCWP, mmHg | 5.2 ± 0.5 | 3.5 ± 0.9* | 2.6 ± 0.5^{†§} | 3.7 ± 0.5^{‡} |
| RAP, mmHg | 1.8 ± 0.4 | 1.1 ± 0.4^{†} | 0.9 ± 0.5^{†§} | 1.3 ± 0.5^{§} |
| PAP, mmHg | 11.7 ± 0.6 | 10.3 ± 0.4*^{‡} | 10.5 ± 0.6^{‡} | 11.3 ± 0.7^{‡} |
| CO, L/min | 3.7 ± 0.3 | 3.4 ± 0.4 | 3.0 ± 0.3^{†} | 2.6 ± 0.3^{†} |
| SVR, | 36.3 ± 4.2 | 39.8 ± 4.7 | 44.2 ± 5.4* | 52.4 ± 4.8^{†} |
| mmHgL⁻¹ · min | | | | |
| PVR, | 1.7 ± 0.2 | 2.0 ± 0.2 | 2.7 ± 0.3^{†} | 3.0 ± 0.3^{†} |
| mmHgL⁻¹ · min | | | | |

CNP Group (n = 7-9)

| | | | | |
|---|---|---|---|---|
| MAP, mmHg | 123 ± 4 | 127 ± 4 | 128 ± 4 | 128 ± 4 |
| PCWP, mmHg | 5.0 ± 0.5 | 4.6 ± 0.5 | 5.6 ± 0.7 | 6.4 ± 0.8* |
| RAP, mmHg | 2.6 ± 0.3 | 2.5 ± 0.3 | 2.9 ± 0.3 | 3.5 ± 0.5^{†} |
| PAP, mmHg | 13.1 ± 0.8 | 12.8 ± 0.8 | 13.2 ± 0.7 | 13.9 ± 0.7 |
| CO, L/min | 3.8 ± 0.3 | 3.3 ± 0.3 | 2.8 ± 0.3^{†} | 2.6 ± 0.1^{†} |
| SVR, | 33.8 ± 2.9 | 39.9 ± 2.7 | 46.9 ± 2.7^{†} | 48.7 ± 2.5^{†} |
| mmHgL⁻¹ · min | | | | |
| PVR, | 1.8 ± 0.2 | 1.9 ± 0.2 | 2.1 ± 0.3 | 2.2 ± 0.4 |
| mmHgL⁻¹ · min | | | | |

Values are means ± SEM.
Comparisons within group vs. pre-infusion, pre-I (mean ± S.E.M., P < 0.05*, <0.01^{†}) and between groups (P < 0.01^{§}).
I, infusion;
MAP, mean arterial pressure;
PAP, pulmonary arterial pressure;
PCWP, pulmonary capillary wedge pressure;
RAP, right atrial pressure;
CO, cardiac output;
SVR, systemic vascular resistance;
PVR, pulmonary vascular resistance.

TABLE 6
Hormonal response to intravenous infusion of CD-NP or CNP

| | | | |
|---|---|---|---|
| Pre-I | 30 min I | 60 min I | Post-I |

CD-NP Group (n = 10)

| | | | | |
|---|---|---|---|---|
| PRA, ng/ml/hr | 6.1 ± 1.4 | 1.8 ± 0.7^{†} | 1.1 ± 0.4^{†} | 7.0 ± 1.6 |
| ANG II, pg/mL | 16.6 ± 3.0 | 7.5 ± 1.8^{†} | 4.4 ± 0.7^{†} | 14.7 ± 2.8 |
| Aldosterone, ng/dL | 21.6 ± 5.0 | 18.2 ± 5.5 | 14.3 ± 4.7 | 18.6 ± 6.4 |

CNP Group (n = 9)

| | | | | |
|---|---|---|---|---|
| PRA, ng/ml/hr | 2.9 ± 1.4 | 1.9 ± 0.6 | 2.5 ± 0.7 | 3.2 ± 1.0 |
| ANG II, pg/mL | 11.7 ± 4.7 | 7.5 ± 1.4 | 10.4 ± 2.8 | 14.4 ± 5.0 |
| Aldosterone, ng/dL | 15.1 ± 4.2 | 21.1 ± 3.8 | 21.4 ± 3.7 | 22.0 ± 4.2 |

Values are means ± SEM.
Comparisons within group vs. pre-infusion, pre-I (mean ± SEM, P < 0.01^{†}) and between groups (P < 0.05^{‡}, <0.01^{§}, <0.001^{∥}).
PRA, plasma renin activity;
ANG II, angiotensin II.

TABLE 7
Statistical Analyses on Data from Human Clinical Trial on CD-NP Comparison of Measurement Variables Between Dosage Groups

| | | | | | | | | | 95% CI | Rank |
|---|---|---|---|---|---|---|---|---|---|---|
| Measurement Variable | Class Variable | n_used | n_miss | mean | sd | median | min | max | For Mean | Sum |
| Plasma cGMP (Baseline) | GROUP = CD-NP | 6 | 0 | 1.40 | 0.36 | 1.450 | 0.80 | 1.80 | (1.02, 1.78) | 0.914 |
| Plasma cGMP (Baseline) | GROUP = Placebo | 4 | 0 | 1.65 | 0.73 | 1.450 | 1.00 | 2.70 | (0.49, 2.81) | |
| Plasma cGMP (At Dosage) | GROUP = CD-NP | 6 | 0 | 7.18 | 2.73 | 8.100 | 3.50 | 10.10 | (4.32, 10.05) | 0.010 |
| Plasma cGMP (At Dosage) | GROUP = Placebo | 4 | 0 | 1.73 | 0.59 | 1.450 | 1.40 | 2.60 | (0.79, 2.66) | |
| Urine cGMP (Baseline) | GROUP = CD-NP | 6 | 0 | 449.14 | 167.22 | 445.005 | 194.27 | 656.36 | (273.66, 624.61) | 0.610 |
| Urine cGMP (Baseline) | GROUP = Placebo | 4 | 0 | 420.42 | 90.14 | 399.550 | 346.42 | 536.16 | (277.16, 563.68) | |
| Urine cGMP (At Dosage) | GROUP = CD-NP | 6 | 0 | 857.61 | 283.85 | 778.580 | 563.38 | 1210.85 | (559.75, 1155.47) | 0.010 |
| Urine cGMP (At Dosage) | GROUP = Placebo | 4 | 0 | 270.85 | 81.86 | 287.735 | 165.71 | 342.21 | (140.74, 400.95) | |
| Na Excretion (Baseline) | GROUP = CD-NP | 6 | 0 | 0.05 | 0.01 | 0.055 | 0.03 | 0.07 | (0.04, 0.07) | 0.762 |
| Na Excretion (Baseline) | GROUP = Placebo | 4 | 0 | 0.05 | 0.01 | 0.050 | 0.04 | 0.07 | (0.03, 0.07) | |
| Na Excretion (At Dosage) | GROUP = CD-NP | 6 | 0 | 0.11 | 0.02 | 0.105 | 0.09 | 0.15 | (0.09, 0.13) | 0.010 |
| Na Excretion (At Dosage) | GROUP = Placebo | 4 | 0 | 0.05 | 0.02 | 0.045 | 0.03 | 0.07 | (0.02, 0.07) | |
| Urine Flowrate (Baseline) | GROUP = CD-NP | 6 | 0 | 1.10 | 0.39 | 1.210 | 0.43 | 1.48 | (0.69, 1.51) | 0.762 |
| Urine Flowrate (Baseline) | GROUP = Placebo | 4 | 0 | 1.25 | 0.34 | 1.210 | 0.90 | 1.69 | (0.72, 1.79) | |
| Urine Flowrate (At Dosage) | GROUP = CD-NP | 6 | 0 | 2.32 | 0.90 | 1.900 | 1.60 | 3.60 | (1.37, 3.27) | 0.476 |
| Urine Flowrate (At Dosage) | GROUP = Placebo | 4 | 0 | 1.58 | 0.77 | 1.665 | 0.69 | 2.29 | (0.35, 2.81) | |
| GFR (Baseline) | GROUP = CD-NP | 6 | 0 | 81.78 | 16.73 | 74.765 | 68.55 | 113.40 | (64.22, 99.33) | 0.762 |
| GFR (Baseline) | GROUP = Placebo | 4 | 0 | 76.47 | 7.12 | 75.125 | 70.35 | 85.27 | (65.16, 87.78) | |
| GFR (At Dosage) | GROUP = CD-NP | 6 | 0 | 84.13 | 18.94 | 76.450 | 71.60 | 121.13 | (64.25, 104.01) | 0.762 |
| GFR (At Dosage) | GROUP = Placebo | 4 | 0 | 77.29 | 6.44 | 76.770 | 70.35 | 85.27 | (67.06, 87.52) | |
| MAP (Baseline) | GROUP = CD-NP | 6 | 0 | 85.38 | 5.37 | 84.950 | 78.45 | 94.08 | (79.74, 91.01) | 0.610 |
| MAP (Baseline) | GROUP = Placebo | 4 | 0 | 86.78 | 2.71 | 85.563 | 85.15 | 90.83 | (82.47, 91.08) | |
| MAP (At Dosage) | GROUP = CD-NP | 6 | 0 | 81.67 | 7.45 | 82.270 | 69.00 | 90.94 | (73.85, 89.49) | 0.762 |
| MAP (At Dosage) | GROUP = Placebo | 4 | 0 | 83.88 | 6.31 | 83.993 | 76.26 | 91.26 | (73.85, 93.9) | |
| Orthostatic Systolic BP | GROUP = CD-NP | 6 | 0 | 116.17 | 9.39 | 113.000 | 105.00 | 130.00 | (106.31, 126.02) | 0.914 |
| (Baseline) Time 0 | | | | | | | | | | |
| Orthostatic Systolic BP | GROUP = Placebo | 4 | 0 | 114.50 | 4.04 | 115.500 | 109.00 | 118.00 | (108.08, 120.92) | |
| (Baseline) Time 0 | | | | | | | | | | |
| Orthostatic Systolic BP | GROUP = CD-NP | 6 | 0 | 110.17 | 10.38 | 108.500 | 97.00 | 129.00 | (99.27, 121.06) | 0.352 |
| (Baseline) Time 4 | | | | | | | | | | |
| Orthostatic Systolic BP | GROUP = Placebo | 4 | 0 | 116.00 | 6.32 | 117.000 | 108.00 | 122.00 | (105.95, 126.05) | |
| (Baseline) Time 4 | | | | | | | | | | |
| Orthostatic Diastolic BP | GROUP = CD-NP | 6 | 0 | 69.00 | 9.53 | 66.500 | 59.00 | 85.00 | (59, 79) | 0.914 |
| (Baseline) Time 0 | | | | | | | | | | |
| Orthostatic Diastolic BP | GROUP = Placebo | 4 | 0 | 66.75 | 3.86 | 66.000 | 63.00 | 72.00 | (60.61, 72.89) | |
| (Baseline) Time 0 | | | | | | | | | | |
| Orthostatic Diastolic BP | GROUP = CD-NP | 6 | 0 | 61.83 | 6.31 | 60.500 | 57.00 | 74.00 | (55.22, 68.45) | 0.610 |
| (Baseline) Time 4 | | | | | | | | | | |
| Orthostatic Diastolic BP | GROUP = Placebo | 4 | 0 | 62.75 | 7.93 | 64.000 | 52.00 | 71.00 | (50.14, 75.36) | |
| (Baseline) Time 4 | | | | | | | | | | |
| Orthostatic Systolic BP | GROUP = CD-NP | 6 | 0 | 113.83 | 12.19 | 117.500 | 97.00 | 126.00 | (101.04, 126.62) | 0.914 |
| (1 Min) Time 0 | | | | | | | | | | |
| Orthostatic Systolic BP | GROUP = Placebo | 4 | 0 | 115.75 | 13.84 | 122.500 | 95.00 | 123.00 | (93.75, 137.75) | |
| (1 Min) Time 0 | | | | | | | | | | |
| Orthostatic Systolic BP | GROUP = CD-NP | 6 | 0 | 108.17 | 8.01 | 107.000 | 98.00 | 118.00 | (99.76, 116.57) | 0.066 |
| (1 Min) Time 4 | | | | | | | | | | |
| Orthostatic Systolic BP | GROUP = Placebo | 4 | 0 | 119.75 | 9.98 | 118.500 | 109.00 | 133.00 | (103.89, 135.61) | |
| (1 Min) Time 4 | | | | | | | | | | |
| Orthostatic Diastolic BP | GROUP = CD-NP | 6 | 0 | 68.00 | 9.74 | 64.500 | 58.00 | 82.00 | (57.78, 78.22) | 0.476 |
| (1 Min) Time 0 | | | | | | | | | | |
| Orthostatic Diastolic BP | GROUP = Placebo | 4 | 0 | 70.50 | 2.38 | 70.500 | 68.00 | 73.00 | (66.72, 74.28) | |
| (1 Min) Time 0 | | | | | | | | | | |
| Orthostatic Diastolic BP | GROUP = CD-NP | 6 | 0 | 63.83 | 6.85 | 63.000 | 57.00 | 74.00 | (56.64, 71.02) | 0.914 |
| (1 Min) Time 4 | | | | | | | | | | |
| Orthostatic Diastolic BP | GROUP = Placebo | 4 | 0 | 63.75 | 7.41 | 63.000 | 56.00 | 73.00 | (51.97, 75.53) | |
| (1 Min) Time 4 | | | | | | | | | | |
| Orthostatic Systolic BP | GROUP = CD-NP | 6 | 0 | 114.17 | 15.37 | 109.500 | 100.00 | 142.00 | (98.04, 130.29) | 0.476 |
| (3 Min) Time 0 | | | | | | | | | | |
| Orthostatic Systolic BP | GROUP = Placebo | 4 | 0 | 119.25 | 10.24 | 118.500 | 110.00 | 130.00 | (102.97, 135.53) | |
| (3 Min) Time 0 | | | | | | | | | | |
| Orthostatic Systolic BP | GROUP = CD-NP | 6 | 0 | 108.17 | 19.08 | 101.000 | 91.00 | 135.00 | (88.14, 128.19) | 0.610 |
| (3 Min) Time 4 | | | | | | | | | | |
| Orthostatic Systolic BP | GROUP = Placebo | 4 | 0 | 114.75 | 16.28 | 115.500 | 96.00 | 132.00 | (88.88, 140.62) | |
| (3 Min) Time 4 | | | | | | | | | | |
| Orthostatic Diastolic BP | GROUP = CD-NP | 6 | 0 | 66.50 | 8.69 | 62.500 | 58.00 | 78.00 | (57.38, 75.62) | 0.476 |
| (3 Min) Time 0 | | | | | | | | | | |
| Orthostatic Diastolic BP | GROUP = Placebo | 4 | 0 | 72.25 | 1.71 | 72.500 | 70.00 | 74.00 | (69.54, 74.96) | |
| (3 Min) Time 0 | | | | | | | | | | |
| Orthostatic Diastolic BP | GROUP = CD-NP | 6 | 0 | 61.00 | 11.15 | 62.000 | 44.00 | 78.00 | (49.3, 72.7) | 0.762 |
| (3 Min) Time 4 | | | | | | | | | | |
| Orthostatic Diastolic BP | GROUP = Placebo | 4 | 0 | 66.50 | 11.45 | 68.500 | 53.00 | 76.00 | (48.31, 84.69) | |
| (3 Min) Time 4 | | | | | | | | | | |
| Plasma Angiotensin II | GROUP = CD-NP | 6 | 0 | 29.17 | 5.19 | 29.500 | 20.00 | 35.00 | (23.72, 34.62) | 0.114 |
| (Pre-Infusion) | | | | | | | | | | |
| Plasma Angiotensin II | GROUP = Placebo | 4 | 0 | 23.25 | 2.22 | 23.000 | 21.00 | 26.00 | (19.73, 26.77) | |
| (Pre-Infusion) | | | | | | | | | | |
| Plasma Angiotensin II | GROUP = CD-NP | 6 | 0 | 23.00 | 3.52 | 23.000 | 18.00 | 29.00 | (19.3, 26.7) | 0.476 |
| (End of Infusion) | | | | | | | | | | |
| Plasma Angiotensin II | GROUP = Placebo | 4 | 0 | 19.75 | 8.18 | 19.500 | 10.00 | 30.00 | (6.75, 32.75) | |
| (End of Infusion) | | | | | | | | | | |
| Aldosterone (Pre-Infusion) | GROUP = CD-NP | 6 | 0 | 21.93 | 2.73 | 20.800 | 19.30 | 25.50 | (19.07, 24.79) | 0.352 |
| Aldosterone (Pre-Infusion) | GROUP = Placebo | 4 | 0 | 20.33 | 4.16 | 18.800 | 17.30 | 26.40 | (13.71, 26.94) | |
| Aldosterone | GROUP = CD-NP | 6 | 0 | 9.50 | 3.16 | 8.400 | 6.20 | 14.90 | (6.19, 12.81) | 0.114 |
| (End of Infusion) | | | | | | | | | | |
| Aldosterone | GROUP = Placebo | 4 | 0 | 13.55 | 4.46 | 13.100 | 9.00 | 19.00 | (6.46, 20.64) | |
| (End of Infusion) | | | | | | | | | | |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for reducing cardiac remodeling in a subject identified as being in need thereof, said method comprising administering to said subject a composition comprising a pharmaceutically acceptable carrier and a polypeptide capable of increasing urinary and plasma cyclic 3′5′ guanosine monophosphate (cGMP) levels in said subject, wherein said composition is administered in an amount effective to alter the level of one or more parameters of cardiac remodeling by at least ten percent as compared to the levels of said one or more parameters prior to administering said composition, and wherein said one or more parameters are selected from the group consisting of cardiac unloading, increased glomerular filtration rate, decreased levels of aldosterone, decreased plasma renin activity, decreased levels of angiotensin II, decreased proliferation of cardiac fibroblasts, decreased left ventricular mass, decreased left ventricular hypertrophy, decreased ventricular fibrosis, increased ejection fraction, decreased left ventricular end systolic diameter, decreased pulmonary wedge capillary pressure, decreased right atrial pressure, and decreased mean arterial pressure.

2. The method of claim 1, wherein said polypeptide is a natriuretic polypeptide.

3. The method of claim 2, wherein said natriuretic polypeptide is a chimeric natriuretic polypeptide comprising (a) the ring structure of a first natriuretic polypeptide or a variant of the ring structure of said first natriuretic polypeptide, and (b) an amino acid sequence from a second natriuretic polypeptide or a variant of said amino acid sequence from said second natriuretic polypeptide.

4. The method of claim 2, wherein said natriuretic polypeptide comprises the amino acid sequence set forth in SEQ ID NO:3, but with one, two, three, four, or five amino acid substitutions relative to the sequence set forth in SEQ ID NO:3.

5. The method of claim 1, wherein said polypeptide is capable of binding to the NPR-B receptor and the NRP-A receptor.

6. The method of claim 1, wherein said polypeptide has an elimination half-life of at least 15 minutes after administration to said subject.

7. The method of claim 1, comprising administering said composition as a continuous intravenous infusion.

8. The method of claim 7, comprising administering said continuous intravenous infusion for one to seven days.

9. The method of claim 1, comprising administering said composition as a continuous intravenous infusion for one to seven days, and subsequently administering said composition subcutaneously for five to 30 days.

10. The method of claim 1, comprising administering said composition as a continuous intravenous infusion at a dose of about 0.1 ng polypeptide/kg body mass/minute to about 30 ng polypeptide/kg body mass/minute, and subsequently administering said composition subcutaneously at a dose of about 10 ng polypeptide/kg body mass/day to about 30 ng polypeptide/kg body mass/day.

11. The method of claim 1, comprising administering said composition as a continuous intravenous infusion at a dose of about 0.1 ng polypeptide/kg body mass/minute to about 30 ng polypeptide/kg body mass/minute for about three hours to about seven days, and subsequently administering said composition subcutaneously at a dose of about 10 ng polypeptide/kg body mass/day to about 30 ng polypeptide/kg body mass/day for about five to about 30 days.

12. The method of claim 1, wherein said subject is identified as having acute heart failure or acute myocardial infarction.

13. The method of claim 12, comprising administering said continuous intravenous infusion beginning at or about the time of reperfusion.

14. The method of claim 12, wherein said composition is administered beginning about three hours after the onset of reperfusion.

15. The method of claim 12, wherein said composition is administered from about three hours to about 12 hours after reperfusion.

16. The method of claim 1, comprising administering said composition at a dose of about 1 ng polypeptide/kg body mass/minute to about 30 ng polypeptide/kg body mass/minute.

17. The method of claim 1, further comprising monitoring said subject for said level of one or more parameters of cardiac remodeling.

18. A composition comprising a pharmaceutically acceptable carrier and a polypeptide, wherein said polypeptide is capable of increasing urinary and plasma cGMP levels in a subject, wherein said composition, when administered to a subject identified as being in need thereof, results in reduced cardiac remodeling, wherein said reduced or inhibited cardiac remodeling is indicated by an alteration in the levels of one or more parameters selected from the group consisting of cardiac unloading, increased glomerular filtration rate, decreased levels of aldosterone, decreased plasma renin activity, decreased levels of angiotensin II, decreased proliferation of cardiac fibroblasts, decreased left ventricular mass, decreased left ventricular hypertrophy, decreased ventricular fibrosis, increased ejection fraction, decreased left ventricular end systolic diameter, decreased pulmonary wedge capillary pressure, decreased right atrial pressure, and decreased mean arterial pressure, and wherein the levels of said one or more parameters are altered by at least ten percent as compared to the levels of said one or more parameters prior to said administration.

19. The composition of claim 18, wherein said polypeptide is a natriuretic polypeptide.

20. The composition of claim 19, wherein said natriuretic polypeptide is a chimeric natriuretic polypeptide comprising (a) the ring structure of first natriuretic polypeptide or a variant of the ring structure of said first natriuretic polypeptide, and (b) an amino acid sequence from a second natriuretic polypeptide or a variant of said amino acid sequence from said second natriuretic polypeptide.

21. The composition of claim 19, wherein said natriuretic polypeptide comprises the amino acid sequence set forth in SEQ ID NO:3, but with one, two, three, four, or five amino acid substitutions relative to the sequence set forth in SEQ ID NO:3.

22. The composition of claim 18, wherein said polypeptide is capable of binding to the NPR-B receptor and the NRP-A receptor.

23. The composition of claim 18, wherein said polypeptide has an elimination half-life of at least 15 minutes after administration to a subject.

24. The composition of claim 18, wherein said natriuretic polypeptide comprises an amino acid sequence that is between 91 and 98 percent identical to the amino acid sequence set forth in SEQ ID NO:3.

25. The composition of claim 18, wherein said natriuretic polypeptide comprises the amino acid sequence of SEQ ID NO:3, but with one, two, three, four, or five amino acid substitutions relative to the sequence set forth in SEQ ID NO:3.

26. The composition of claim 18, wherein said subject is identified as having acute heart failure or acute myocardial infarction.

27. The composition of claim 18, wherein said pharmaceutical carrier is normal saline or dextrose and water.


SEQUENCE LISTING (17 sequences)

SEQ ID NO: 1 - protein, 22 aa, Homo sapiens
GLSKGCFGLKLDRIGSMSGLGC

SEQ ID NO: 2 - protein, 15 aa, Dendroaspis angusticeps
PSLRDPRPNAPSTSA

SEQ ID NO: 3 - protein, 37 aa, Artificial Sequence
  Synthetic
GLSKGCFGLKLDRIGSMSGLGCPSLRDPRPNAPSTSA

SEQ ID NO: 4 - protein, 27 aa, Artificial Sequence
  Synthetic
GLSKGCFGLKLDRIGSMSGLGCGKSLG

SEQ ID NO: 5 - protein, 17 aa, Homo sapiens
CFGLKLDRIGSMSGLGC

SEQ ID NO: 6 - protein, 10 aa, Homo sapiens
TAPRSLRRSS

SEQ ID NO: 7 - protein, 5 aa, Homo sapiens
NSFRY

SEQ ID NO: 8 - protein, 32 aa, Artificial Sequence
  Synthetic
TAPRSLRRSSCFGLKLDRIGSMSGLGCNSFRY

SEQ ID NO: 9 - protein, 37 aa, Artificial Sequence
  Synthetic
PLSKGCFGLKLDRIGSMSGLGCPSLRDPRPNAPSTSA

SEQ ID NO: 10 - protein, 37 aa, Artificial Sequence
  Synthetic
GISKGCFGLKLDRIGSMSGLGCPSLRDPRPNAPSTSA

SEQ ID NO: 11 - protein, 37 aa, Artificial Sequence
  Synthetic
GLSKGCFGLKLDRIGSMSGLGCPSLRDPRPNAPSTTA

SEQ ID NO: 12 - protein, 37 aa, Artificial Sequence
  Synthetic
GLSKGCFGLKLDRIGSMSGLGCPSLRDPRPNAPSTSV

SEQ ID NO: 13 - protein, 37 aa, Artificial Sequence
  Synthetic
GLTKGCFGLKLDRIGSMSGLGCPSLRDPRPNAPSTSA

SEQ ID NO: 14 - protein, 37 aa, Artificial Sequence
  Synthetic
GLSRGCFGLKLDRIGSMSGLGCPSLRDPRPNAPSTSA

SEQ ID NO: 15 - protein, 37 aa, Artificial Sequence
  Synthetic
GLSKGCFGLKLDRIGSMSGLGCPSLRDPRPNAPSSSA

SEQ ID NO: 16 - protein, 37 aa, Artificial Sequence
  Synthetic
GLSKGCFGLKLDRIGSMSGLGCPSLRDPRPNAPTTSA

SEQ ID NO: 17 - protein, 38 aa, Dendroaspis angusticeps
EVKYDPCFGHKIDRINHVSNLGCPSLRDPRPNAPSTSA